SEQUENCE LISTING (32 sequences)

SEQ ID NO: 1 - DNA, 20 bp, Artificial Sequence
  synthetically generated primers
tctttctccagctgttcacc

SEQ ID NO: 2 - DNA, 20 bp, Artificial Sequence
  synthetically generated primers
atgatgagaggcagcaagag

SEQ ID NO: 3 - DNA, 19 bp, Artificial Sequence
  synthetically generated primers
tccgccccttctgccgatg

SEQ ID NO: 4 - DNA, 21 bp, Artificial Sequence
  synthetically generated primers
cacggaaggccatgccagtga

SEQ ID NO: 5 - DNA, 20 bp, Artificial Sequence
  synthetically generated primers
tgttttgctccctggaaggc

SEQ ID NO: 6 - DNA, 23 bp, Artificial Sequence
  synthetically generated primers
agtcactgctgtttgtgctctcc

SEQ ID NO: 7 - DNA, 24 bp, Artificial Sequence
  synthetically generated primers
acctgtcctgtgtaatgaaagacg

SEQ ID NO: 8 - DNA, 19 bp, Artificial Sequence
  synthetically generated primers
tgggtattgcttgggatcc

SEQ ID NO: 9 - DNA, 23 bp, Artificial Sequence
  synthetically generated primers
caagagacttccatccagttgcc

SEQ ID NO: 10 - DNA, 24 bp, Artificial Sequence
  synthetically generated primers
catttccacgatttcccagagaac

SEQ ID NO: 11 - DNA, 20 bp, Artificial Sequence
  synthetically generated primers
cagccttgcagaaaagagag

SEQ ID NO: 12 - DNA, 20 bp, Artificial Sequence
  synthetically generated primers
ggaagtgggtgcagttattg

SEQ ID NO: 13 - DNA, 24 bp, Artificial Sequence
  synthetically generated primers
tgtggagagacatcagagtcaatg

SEQ ID NO: 14 - DNA, 21 bp, Artificial Sequence
  synthetically generated primers
cgtcaatcaaccctttcatcc

SEQ ID NO: 15 - DNA, 21 bp, Artificial Sequence
  synthetically generated primers
ctatggctgctgctgctattg

SEQ ID NO: 16 - DNA, 23 bp, Artificial Sequence
  synthetically generated primers
ggctcttcctttctcctgtcaac

SEQ ID NO: 17 - DNA, 24 bp, Artificial Sequence
  synthetically generated primers
tgtggctaccagagataactgttg

SEQ ID NO: 18 - DNA, 20 bp, Artificial Sequence
  synthetically generated primers
atgtcttccagcgttcggag

SEQ ID NO: 19 - DNA, 25 bp, Artificial Sequence
  synthetically generated primers
ggagatacctggagtaatcacactg

SEQ ID NO: 20 - DNA, 20 bp, Artificial Sequence
  synthetically generated primers
tagccacggtgctcacaagg

SEQ ID NO: 21 - DNA, 22 bp, Artificial Sequence
  synthetically generated primers
agcagcccagcgacaagaaaag

SEQ ID NO: 22 - DNA, 23 bp, Artificial Sequence
  synthetically generated primers
gcagtcagaaccatcatccgaag

SEQ ID NO: 23 - DNA, 20 bp, Artificial Sequence
  synthetically generated primers
tagcctaagacctgcccctc

SEQ ID NO: 24 - DNA, 20 bp, Artificial Sequence
  synthetically generated primers
accgtgaacacagcctcctg

SEQ ID NO: 25 - DNA, 23 bp, Artificial Sequence
  synthetically generated primers
ggacaatgacaacgacaagttcg

SEQ ID NO: 26 - DNA, 20 bp, Artificial Sequence
  synthetically generated primers
ataccagcgggttttccagg

SEQ ID NO: 27 - DNA, 20 bp, Artificial Sequence
  synthetically generated primers
tatttgggggaacaggttgc

SEQ ID NO: 28 - DNA, 22 bp, Artificial Sequence
  synthetically generated primers
caagtcactctggttagccgtg

SEQ ID NO: 29 - DNA, 22 bp, Artificial Sequence
  synthetically generated primers
agaagaggcactcccccaaaag

SEQ ID NO: 30 - DNA, 24 bp, Artificial Sequence
  synthetically generated primers
ttcagtagacagaagagcgtggtg

SEQ ID NO: 31 - protein, 28 aa, Artificial Sequence
  synthetically generated peptides
GHRPLDKKREEAPSLRPAPPPISGGGYR

SEQ ID NO: 32 - protein, 28 aa, Artificial Sequence
  synthetically generated peptides
RYGGGSIPPPAPRLSPAEERKKDLPRHG

(19) United States
(12) Patent Application Publication
(10) Pub. No.: US 2013/0324469 A1
(43) Pub. Date: Dec. 5, 2013

(54) FIBRINOGEN AND KIDNEY DAMAGE

(71) Applicant: Vishal S. Vaidya, Cambridge, MA (US)

(72) Inventor: Vishal S. Vaidya, Cambridge, MA (US)

(73) Assignee: THE BRIGHAM AND WOMEN'S HOSPITAL, INC., Boston, MA (US)

(21) Appl. No.: 13/992,425

(22) PCT Filed: Dec. 7, 2011
(86) PCT No.: PCT/US2011/063709

§ 371 Date: Aug. 19, 2013

Related U.S. Application Data
(60) Provisional application No. 61/421,428, filed on Dec. 9, 2010.

(51) Int. Cl. G01N 33/86; A61K 38/36

(52) U.S. Cl. CPC G01N 33/86; A61K 38/363
USPC 514/13.6; 506/9

(57) ABSTRACT

Methods for diagnosing and monitoring acute kidney injury (AKI) based on urinary fibrinogen levels, and methods for treating AKI using a Bbeta15-42 peptide.

### CLAIM OF PRIORITY

This application claims the benefit of U.S. Provisional Application Ser. No. 61/421,428, filed on Dec. 9, 2010, which is incorporated herein by reference in its entirety.

### FEDERALLY SPONSORED RESEARCH OR DEVELOPMENT

This invention was made with Government support under Grant No. ES016723 and ES17543 awarded by the National Institutes of Health. The Government has certain rights in the invention.

### TECHNICAL FIELD

This invention relates to methods for diagnosing, monitoring, and treating kidney damage.

### BACKGROUND

Acute kidney injury (AKI) is a common disorder that portends adverse outcomes in critically ill and non-critically ill hospitalized patients (Barrantes et al., Mayo Clin Proc 2009; 84: 410-416; Chertow et al., J Am Soc Nephrol 2005; 16: 3365-3370; Uchino et al., JAMA 2005; 294: 813-818). Serum creatinine (SCr) and blood urea nitrogen (BUN), that are most commonly used to detect kidney toxicity/injury in preclinical and clinical studies and in routine clinical care have severe limitations relating to their sensitivity and specificity (Vaidya et al., Annu Rev Pharmacol Toxicol 2008; 48: 463-493; Vaidya et al., Nat Biotechnol 2010; 28: 478-485). Furthermore, BUN and SCr concentration render a very delayed signal even after considerable kidney injury and this delay in the diagnosis of AKI prevents timely patient-management decisions, such as withdrawal or reduction in dose of the offending agent or administration of agents to mitigate the injury (Bonventre et al., Nat Biotechnol 2010; 28: 436-440; Siew et al., J Am Soc Nephrol 2011; 22: 810-820).

Kidney disease is a major public health concern receiving increased global attention owing to the significantly increased prevalence and high mortality rates (Eckardt and Kasiske, Nat Rev Nephrol. 2009; 5(11):650-657; Szczech et al., J Am Soc Nephrol. 2009; 20(3):453-455). Renal ischemia/reperfusion (I/R) accounts for the majority of AKI in humans. Studies suggest that damage to the renal microvascular architecture and deterioration of the angiogenic response constitutes the early steps in the complex multiple pathways involved in both early and chronic ischemic renal injury (Lerman and Chade, Curr Opin Nephrol Hypertens. 2009; 18(2):160-165). Restoration of blood flow to the site of injured tissue is crucial for developing a successful repair response that involves the surviving dedifferentiated cells spreading over the denuded basement membrane, undergoing mitogenesis and ultimately re-differentiating to re-establish and restore functional integrity of the nephron (Basile, J Am Soc Nephrol. 2007; 18(1):7-9; Reinders et al., J Am Soc Nephrol. 2006; 17(4):932-942). While these processes are well described at the pathological level, very little is known about the cellular and molecular mechanisms of action of blood proteins within the kidney and their contribution to pathogenesis of renal disease.

### SUMMARY

The present invention is based, at least in part, on the discovery that the presence of elevated levels of fibrinogen is associated with AKI in a subject. In addition, administration of Bβ₁₅₋₄₂, a naturally occurring 28 amino acid long product cleaved from fibrin fragments, is demonstrated to have therapeutic efficacy in an animal model of AKI.

Thus, in a first aspect, the invention provides methods for detecting the presence of acute kidney injury (AKI) in a subject. The methods include determining a level of fibrinogen in a sample comprising urine from a subject; and comparing the level of fibrinogen in the sample to a reference level of fibrinogen, wherein the level of fibrinogen as compared to the reference level indicates whether the subject has AKI.

In some embodiments, the methods include selecting a subject who is suspected of or at risk for having AKI, or who has one or more risk factors for developing AKI. In some embodiments, the subject has minimal change disease.

In some embodiments, determining a level of fibrinogen comprises determining a level of whole fibrinogen protein, and/or one, two, or all of the α, β and γ chains of fibrinogen, and/or one or more fibrin derived peptides.

In some embodiments, the reference level represents a level of fibrinogen in a subject who does not have AKI, and the presence of a level of fibrinogen above the reference level indicates that the subject has AKI.

In some embodiments, the methods include administering a treatment for AKI to a subject who has a level of fibrinogen above the reference level.

In another aspect, the invention provides method of selecting a treatment for a subject. The methods include determining a level of fibrinogen in a sample comprising urine of a subject; comparing the level of fibrinogen in the sample to a reference level of fibrinogen; and selecting a treatment for acute kidney injury (AKI) for a subject who has a level of fibrinogen that is above the reference level.

In some embodiments, the methods include selecting a subject who is suspected of or at risk for having AKI, or who has one or more risk factors for developing AKI. In some embodiments, the subject has minimal change disease.

In some embodiments, determining a level of fibrinogen comprises determining a level of whole fibrinogen protein, and/or one, two, or all of the α, β and γ chains of fibrinogen, and/or one or more fibrin derived peptides.

In some embodiments, the reference level represents a level of fibrinogen in a subject who does not have AKI, and the presence of a level of fibrinogen above the reference level indicates that the subject has AKI.

In some embodiments, the methods include administering the selected treatment for AKI to a subject who has a level of fibrinogen above the reference level.

In an additional aspect, the invention provides methods for treating a subject. The methods include determining a level of fibrinogen in a sample comprising urine of a subject; comparing the level of fibrinogen in the sample to a reference level of fibrinogen; and administering a treatment for acute kidney injury (AKI) to the subject based on the presence of a level of fibrinogen that is above the reference level.

In a further aspect, the invention provides methods for treating a subject who has acute kidney injury. The methods include administering a composition comprising a therapeutically effective amount of a fibrin derive peptide, e.g., a peptide comprising Bβ₁₅₋₄₂ (GHRPLDKKREEAPSLRPAPPPISGGGYR (SEQ ID NO:31)), to a subject who is in need of, or who has been determined to be in need of, such treatment. In some embodiments, the subject has been identified as having or being at risk for AKI, e.g., using a method known in the art or described herein.

In some embodiments, the peptide comprises Bβ₁₅₋₄₂ peptide fused to a cell-penetrating peptide.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a set of bar graphs showing real time PCR analysis in kidney (left and center columns) and liver (right column) tissues for Fgα, Fgβ and Fgγ chains, normalized using a housekeeping gene (Gapdh), and fold change determined over sham group. (n=5/group). * represents p<0.05 as determined by one way ANOVA in comparison with sham rats. Fibrinogen (Fgα, Fgβ and Fgγ chains) was significantly up regulated in kidney cortex and medulla of male Wistar rats following 20 minutes bilateral renal ischemia/reperfusion injury as compared to sham surgery.

FIG. 1B is a pair of bar graphs showing serum creatinine and BUN measurements in the plasma of Male Wistar rats subjected to 20 min bilateral ischemia/reperfusion.

FIG. 1C is a bar graph showing RT-PCR of Kim-1 gene expression in cortex and medulla in Male Wistar rats subjected to 20 min bilateral ischemia/reperfusion healthy versus. n=5/group. * represents p<0.05 as determined by one way ANOVA in comparison with sham rats. Bar represents 100 μm.

FIG. 1D is a set of nine bar graphs showing the results of Real Time PCR analysis for Fgα, Fgβ and Fgγ chain gene expression in heart, lung and spleen collected from male Wistar rats subjected to 20 min bilateral ischemia, followed by reperfusion for 6, 24, 72 and 120 h and compared with healthy (sham) rats (n=5/group/time point).

FIG. 2A is a set of three bar graphs showing the results when urinary fibrinogen (Fg) was compared with tubular injury biomarkers N-acetyl-β-glucosaminidase (NAG) and kidney injury molecule-1 (Kim-1) in rats subjected to 20 minutes bilateral renal ischemia/reperfusion injury (n=5/group). * represents p<0.05 as determined by Student's t test in comparison with sham rats.

FIG. 2B is a graph showing urinary Fg measured in a human cross-sectional study with clinically established multifactorial AKI (n=25) versus healthy volunteers (n=25). Magenta line and corresponding number marked by arrow indicates a threshold cut off value at 95% specificity. There was a significant increase in urinary fibrinogen after kidney injury in rats and humans.

FIG. 2C is a line graph showing Receiver Operating Curves (ROCs) comparing the sensitivity and specificity of urinary Fg, NAG and KIM-1 to distinguish patients with acute kidney injury (AKI) or chronic kidney disease (CKD) from healthy volunteers.

FIG. 2D is a bar graph showing plasma levels of fibrinogen measured in male Wistar rats that did not undergo any surgery, sham rats and rats subjected to 20 min bilateral ischemia followed by reperfusion (n=5/group/time point). * represents p<0.05 as determined by one way ANOVA in comparison with sham rats.

FIG. 2E is a pair of dot graphs showing urinary N-acetyl-β-glucosaminidase (NAG) and kidney injury molecule-1 (KIM-1) measured in a human cross-sectional study with clinically established multifactorial acute kidney injury (AKI) or chronic kidney disease (CKD) (n=25) versus healthy volunteers (n=25). Horizontal lines and corresponding number marked by arrow indicate a threshold cut off value at 95% specificity (2.62 U NAG/g Cr, and 889.2 pg KIM-1/mg Cr).

FIG. 3 is a set of four bar graphs showing that Bβ₁₅₋₄₂ peptide protects mice from renal ischemia/reperfusion (I/R) injury. Male C57Bl6 mice were subjected to 27 minutes bilateral renal ischemia/reperfusion injury or sham surgery and 3.6 mg/kg of Bβ₁₅₋₄₂ peptide or random peptide was administered intravenously 1 min following reperfusion. The infarct size following ischemia was significantly smaller in the Bβ₁₅₋₄₂ peptide administered mice compared to mice administered random peptide. Serum creatinine (SCr), blood urea nitrogen (BUN) as indicators of renal dysfunction and urinary levels of fibrinogen (Fg) and kidney injury molecule-1 (Kim-1) as indicators of kidney injury were measured at 24 and 48 h in all the groups. (n=5/group of sham, n=10/group at 24 hours and n=5/group at 48 hours).* represents p<0.05 as determined by one way ANOVA in comparison with sham mice.

FIG. 4A is a set of six bar graphs showing the results of Real Time-PCR for candidate markers of inflammation in kidney of mice subjected to 27 min bilateral ischemia, followed by reperfusion for 24 and 48 hours that were administered either 3.6 mg/kg of Bβ15-42 or random peptide intravenously (n=5/group/time point). * represents p<0.05 as determined by Student's t-test between the two groups within the same time point.

FIG. 4B is a bar graph showing a quantification of myeloperoxidase immunostaining in kidneys of Bβ15-42 and random peptide administered groups of mice at 24 and 48 h post 27 min ischemia.

FIGS. 4C-4D are bar graphs showing the results when paraffin embedded kidneys of mice subjected to 27 min bilateral ischemia/reperfusion that were administered either Bβ₁₅₋₄₂ or random peptides were compared at 24 and 48 h for numbers of apoptotic cells by TUNEL assay (4C), or numbers of proliferative cells determined by Ki67 positive staining cells (4D). * represents p<0.05 as determined by Student's t-test between the two groups within the same time point. The results indicate that Bβ₁₅₋₄₂ peptide aids in the resolution of injury by decreasing necrosis/apoptosis and inducing rapid tissue regeneration.

FIG. 4E is a bar graph showing cell proliferation measured by bromodeoxyuridine (5-bromo-2-deoxyuridine (BrdU) uptake, by LLC-PK1 cells, 48 h post onset of hypoxia at 450 nm wavelength. Absorbance obtained from untreated cells was taken as 100% (n=6 wells/group). * represents p<0.05 as determined by Student's t-test between the two groups.

FIGS. 5A-C show the results of analysis of serum creatinine and BUN (5A), urinary Fg (5B-C), and urinary Kim-1 and NAG (5C) in male Wistar rats that underwent bilateral renal ischemia for 30 min following reperfusion for 24, 72 or 120 h compared to rats underwent sham surgery. Data are presented as individual animals and color-coded according to histopathology scores of the acute tubular injury in the kidney. The black line indicates median value of four individual animals per group. The level of acute tubular injury was scored as 0 (none), 1 (mild and limited), or 3 (widespread and severe). Statistical analysis was performed by Student's t-test (*p<0.05).

FIGS. 6A-C show the results of determination of serum creatinine and BUN (6A), urinary Fg (6B-C), and urinary Kim-1 and NAG (6C) in male Balb/C mice treated with a single ip injection of 20 mg/kg cisplatin for 0, 24, 48 and 72 h, respectively. Data are presented as individual animals and color-coded according to histopathology scores of acute tubular injury in the kidney. The mean of five individual animals is indicated as black line. Statistical analysis was performed by Student's t-test (*p<0.05).

FIG. 7 is a line graph showing levels of Serum creatinine (SCr), KIM-1, Fg, and NAG in 31 patients undergoing abdominal aortic aneurysm (AAA) repair, before and at various time points post-operatively.

FIG. 8 is a bar graph showing the results of a comparison of fibrinogen immunostaining patterns in human kidney biopsies in patients without (“normal”) and with acute kidney injury (AKI). Shown are average fibrinogen immunostaining intensity scores (+/−standard error mean) classified by luminal, apical vs. interstitial patterns.

FIG. 9A is a bar graph showing total urinary protein (left Y-axis) and Serum creatinine (right Y-axis) in patients with minimal change disease, without (no AKI) and with (AKI) acute kidney injury represented as average+/−standard error mean. Statistical analysis was performed by Student's t-test (*p<0.05)

FIG. 9B is a bar graph showing average fibrinogen immunostaining intensity scores (+/−standard error mean) in patients with minimal change disease, without (no AKI) and with (AKI) acute kidney injury, classified by luminal, apical vs. interstitial patterns. Statistical analysis was performed by Student's t-test (*p<0.05)

### DETAILED DESCRIPTION

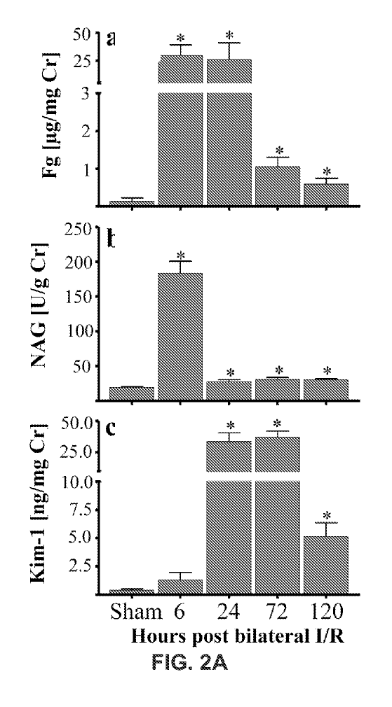

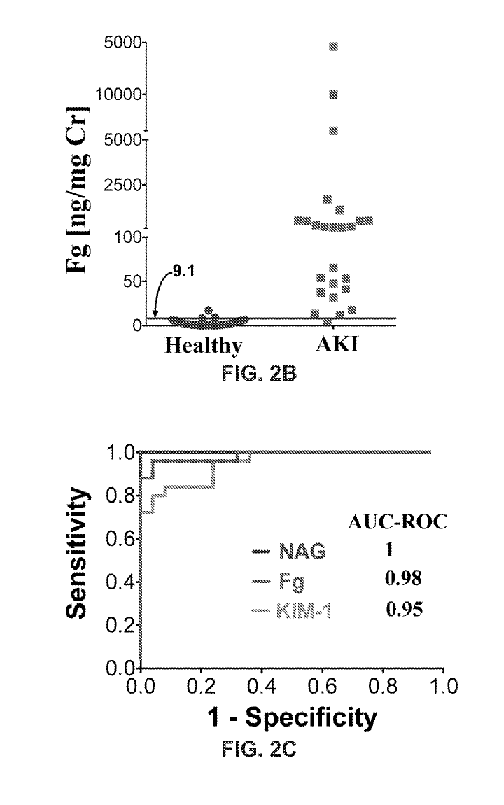

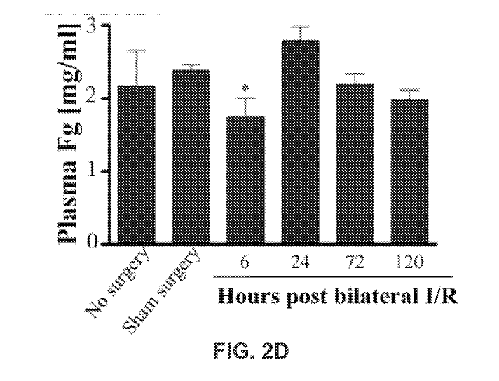

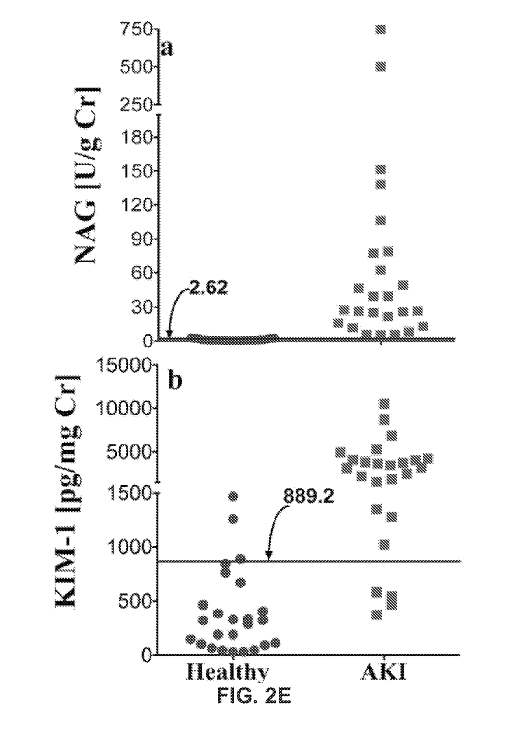

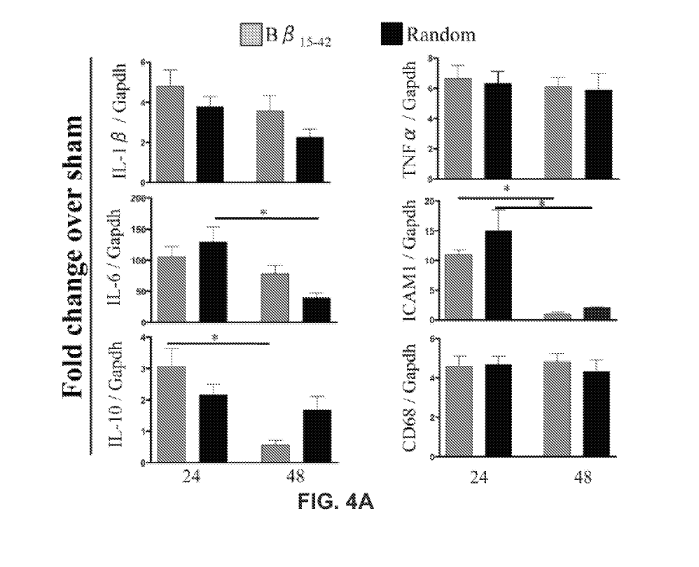

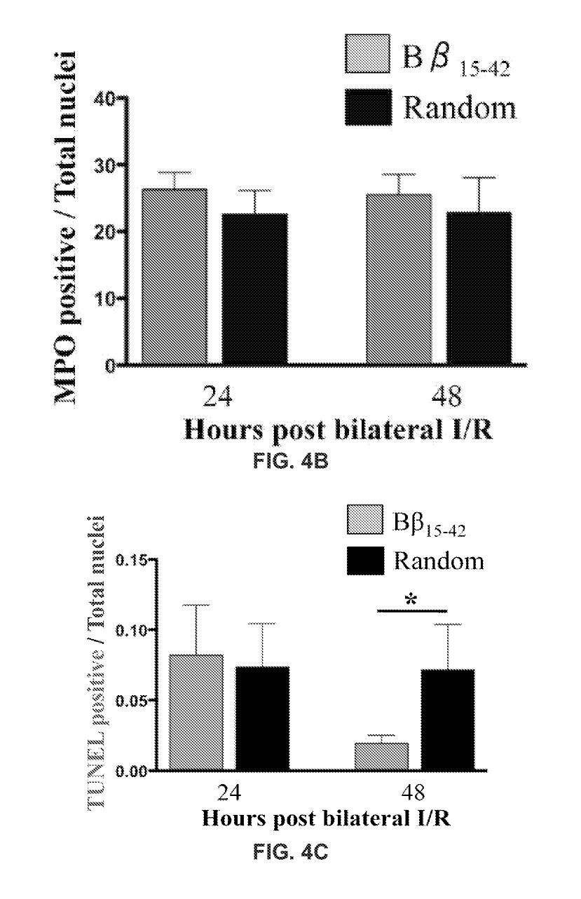

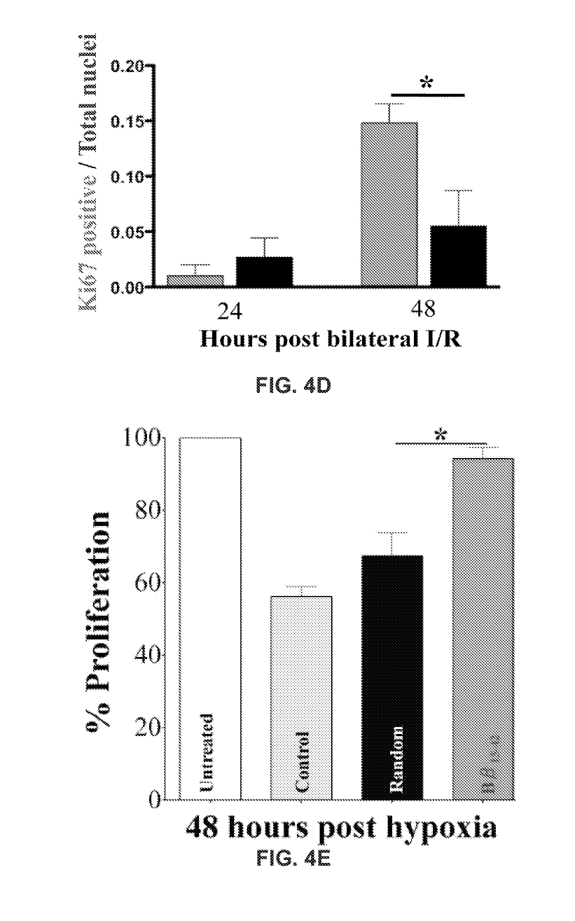

Although it has been recognized that progressive kidney disease is characterized by gradual deterioration of the renal endothelium, which correlates with the development of tubulointerstitial injury, fibrosis and glomerulosclerosis (Lerman and Chade, Curr Opin Nephrol Hypertens. 2009; 18(2):160-16) there has been little effort to characterize the regulatory role of blood proteins in pathophysiology of AKI. As shown herein, i) in whole genome expression profiling studies Fgβ and Fgγ chains are amongst the highly up regulated genes after 24 hr of ischemic injury both in kidney cortex and medulla; ii) Fg serves as an effective safety and efficacy biomarker for kidney injury not only because of the marked increase in urinary Fg following kidney damage (FIGS. 2A-C), but also due to reduced levels upon Bβ₁₅₋₄₂ peptide mediated protection from kidney injury (FIGS. 4C-D), demonstrating responsiveness to both injury and recovery; and iii) Fg derived Bβ₁₅₋₄₂ administration protects mice from I/R induced kidney injury by aiding kidney tissue repair thus demonstrating for the first time its therapeutic potential in AKI. These findings not only highlight the important role of Fg in renal tissue injury and repair, but also offer a therapeutic strategy to enhance kidney regeneration as opposed to simply preventing further injury or deleterious inflammation in the damaged tissue.

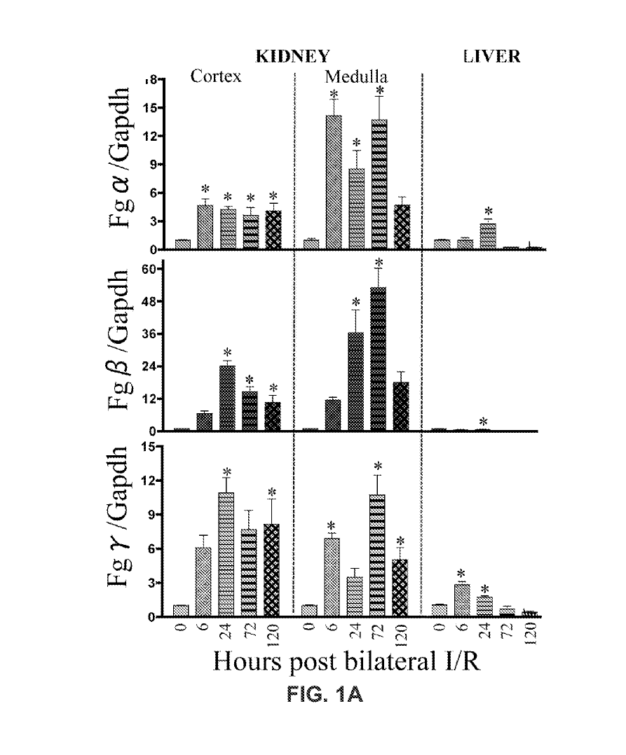

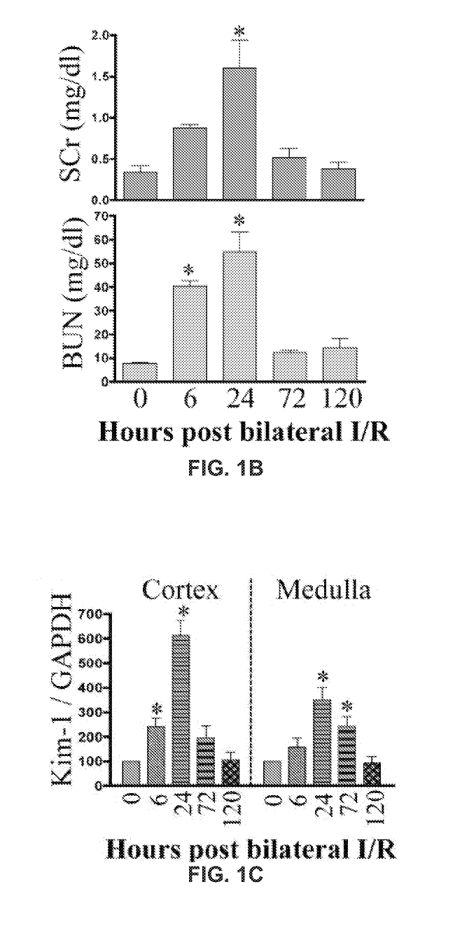

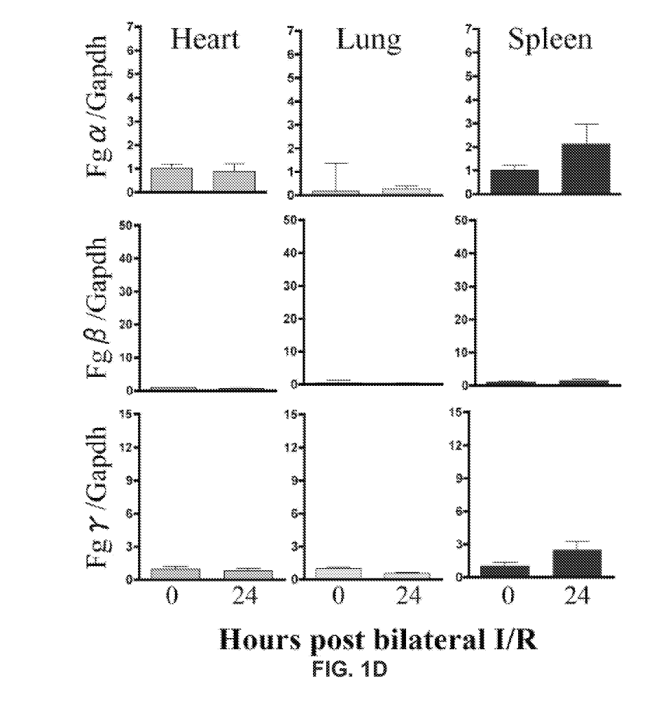

The presence of Fgα, Fgβ, and Fgγ chain transcripts in the kidney at baseline (Baumhueter et al., Genes Dev. 1990; 4(3):372-379) as well as its up regulation following nephrotoxicity (Thukral et al., Toxicol Pathol. 2005; 33(3):343-35) or brain death induced vascular endothelial activation in kidneys (Morariu et al., Am J. Transplant. 2008; 8(5):933-941; Schuurs et al., Am J. Transplant. 2004; 4(12):1972-1981) has been observed before. As described herein, there were distinct expression patterns of Fgα, Fgβ, and Fgγ chains in the renal tubular epithelial cells, glomeruli and interstitium at baseline and during the regeneration in the injured kidney. The increased Fg expression following injury can potentially be a consequence of plasma leakage due to organ damage, as seen after spinal cord injury (Schachtrup et al., Proceedings of the National Academy of Sciences. 2007; 104(28):11814-11819), but the observation of detectable transcript levels of Fgα, Fgβ, and Fgγ chains (FIG. 1A) and corresponding immunoreactivity of all three chains as well as whole Fg molecule in sham rats and in patient without any evidence of tubular injury suggests that the protein could be potentially synthesized and assembled in the kidney.

Plasma Fg has been associated with vascular disease in numerous epidemiological studies (Reinhart W H, Vasc Med. 2003; 8(3):211-216). Although no increase in plasma Fg levels was seen following I/R injury (FIG. 2D), urinary Fg levels increased massively as early as 6 hours following I/R injury that decreased over time, but remained modestly elevated during the resolution phase of injury (FIG. 2A-C). In 1974, Naish et al (Naish et al., Br Med J. 1974; 1(5907):544-546) reported higher levels of urinary fibrin degradation products (FDP) in patients with glomerulonephritis. Subsequently, urinary FDP were shown to be able to make a diagnosis of 25 out of 26 acute rejection episodes at least 24 h before deterioration in renal function and the elevation of FDP preceded the rise in NAG in 9 out of 11 patients (Garcia et al., Proc Eur Dial Transplant Assoc. 1975; 11:311-319).

In a cross sectional study of individuals with and without kidney damage, urinary Fg performed very well in differentiating between patients with and without AKI/CKD with ROC of 0.98. The sensitivity and specificity of urinary Fg was comparable to the other more advanced biomarkers of AKI such as NAG and KIM-1(Vaidya et al., Kidney Int. 2009; 76(1):108-114; Vaidya et al., Nat Biotechnol. 2010; 28(5):478-485; Vaidya et al., Clinical and Translational Science. 2008; 1(3):200-208; Vaidya et al, Kidney Int. 2010).

The current assay is a sandwich ELISA based luminex assay using two polyclonal antibodies against Fg protein and therefore it will be interesting to use a more targeted approach like liquid chromatography-multiple reaction monitoring/mass spectrometry (LC-MRM/MS) to identify whether there is a predominant excretion of Fgβ chain polypeptides in the urine following kidney injury that would correlate with the highest Fgβ chain mRNA levels in the kidney.

The therapeutic efficacy of Fgβ chain derived peptide (Bβ₁₅₋₄₂) was tested in mice subjected to bilateral renal I/R injury and Bβ₁₅₋₄₂ substantially reduced acute tubular injury. Bβ₁₅₋₄₂ is a naturally occurring, 28 amino acid long product, cleaved from fibrin fragments and at suprapharmacological doses, it has shown to protect from myocardial infarction (Petzelbauer et al., Nat Med. 2005; 11(3):298-304) and acute lung injury (Matt et al., Am J Respir Crit Care Med. 2009; 180(12):1208-1217; Groger et al., PLoS One. 2009; 4(4):e5391) in animal models. In a multicentered phase IIa clinical trial with Bβ₁₅₋₄₂ peptide administration, successful protective effects were seen in patients with acute myocardial infarction, whose endothelial barrier integrity had not been compromised (Hallen et al., EuroIntervention;5(8):946-952; Atar et al., J Am Coll Cardiol. 2009; 53(8):720-729). The peptide has also been shown to be vasculoprotective in models of vascular leak in a Fyn-dependent pathway (Groger et al., PLoS One. 2009; 4(4):e5391). Bβ₁₅₋₄₂ has been shown to mediate platelet spreading, proliferation, capillary tube formation and Von Willebrand Factor release and has a binding site for heparin (Groger et al., PLoS One. 2009; 4(4):e5391; Mosesson, J Thromb Haemost. 2005; 3(8):1894-1904). Bβ₁₅₋₄₂ also has low affinity interactions with VE-Cadherin, efficiently disrupting the interaction between Fg with its receptors VE-Cadherin on endothelial cells, thereby stabilizing endothelial barriers, which in turn elicits beneficial anti-inflammatory properties (Petzelbauer et al., Nat Med. 2005; 11(3):298-304. Prepublished on 2005/02/22 as DOI nm1198 [pii] 10.1038/nm1198; Groger et al., PLoS One. 2009; 4(4):e5391).

A unique mechanism of Bβ₁₅₋₄₂ peptide mediated protection in vivo and in vitro results in increased proliferation of renal tubular epithelial cells resulting in decreased necrosis and apoptosis following damage (FIG. 4C-D). Amongst the three chains of Fg, Fgβ chain transcript levels increased the highest (˜50 fold) at 72 h (FIG. 1B) which is the peak of regeneration in this model (Sabbahy et al., Wiley Interdiscip Rev Syst Biol Med. 2010), further underscoring the finding that Fg is up regulated in the kidney as a protective mechanism to aid in regeneration. Kidney regeneration, after an episode of AKI, is a major determinant of outcome for patients with AKI and therefore the use of Bβ₁₅₋₄₂ peptide offers a novel therapy to improve the rate or effectiveness of the tissue repair process after ischemic kidney damage.

In summary, this study provides new opportunities for the use of Fg in diagnosis, prevention, and therapeutic interventions in kidney disease.

Acute Kidney Injury (AKI)

Acute kidney injury (AKI) is most frequently caused by ischemia, sepsis or nephrotoxic insults to the kidney. Clinically AKI is characterised by a rapid reduction in kidney function resulting in a failure to maintain fluid, electrolyte and acid-base homoeostasis. Several definitions and staging system have been proposed for AKI (definitions proposed by the Acute Dialysis Quality Initiative (ADQI), and the Acute Kidney Injury Network (AKIN); RIFLE, Bellomo et al., Crit. Care 2004; 8: R204-R212), see, e.g., Kellum et al., Crit Care Med 2008; 36[Suppl.]:S141-S145, and newer guidelines are coming into more general use (Kidney Disease: Improving Global Outcomes. Clinical practice guideline on acute kidney injury. 2011). Once a subject has developed AM, presently the therapeutic options are limited, with the most common treatment being renal replacement therapy (RRT).

Acute kidney injury can be diagnosed when one of the following criteria is met:
(1) Serum creatinine rises by ≧26 μmol/L within 48 hours or
(2) Serum creatinine rises ≧1.5 fold from the reference value, which is known or presumed to have occurred within one week or
(3) urine output is <0.5 ml/kg/hr for >6 consecutive hours

The reference serum creatinine should be the lowest creatinine value recorded within 3 months of the event. If a reference serum creatinine value is not available within 3 months and AKI is suspected, the serum creatinine should be repeated within 24 hours, and a reference serum creatinine value can be estimated from the nadir serum creatinine value if patient recovers from AKI.

AKI risk factors include: age >75 yrs; chronic kidney disease (CKD, eGFR<60 mls/min/1.73 m2); cardiac failure; atherosclerotic peripheral vascular disease; liver disease; diabetes mellitus; hypervolemia; and nephrotoxic medications (such as non-steroidal anti-inflammatory drugs and aminoglycosides, and radiological contrast agents). See, e.g., Liaño et al., Kidney International 1996; 50:811-818; Lines and Lewington, Clin Med. 2009 June; 9 (3):273-7.

For additional information about AKI and treatments for AKI, see the UK Renal Association Clinical Practice Guideline “Acute Kidney Injury,” Lewington and Kanagasundaram, March 2011, available at renal.org/libraries/guidelines/acute_kidney_injury_-_final_version_{—}08 march 2011.sflb.ashx.

Methods of Diagnosis

Described herein are methods for the diagnosis of acute kidney injury, or AKI. The methods include measuring, in a urine sample from a subject, levels of fibrinogen (Fg). Fibrinogen (factor I) is a soluble plasma glycoprotein, synthesised by the liver, that is converted by thrombin into fibrin during blood coagulation. The methods can include measuring whole or total Fg, and/or one, two, or all of the α, β and γ chains of fibrinogen individually, or fibrin derived peptides.

Fibrin derived peptides (also known as fibrinogen split products) could be derived from any of the three chains, and can include fibrin fragment D (Biochim Biophys Acta 1982; 718(2):177); fibrinmonomer (Thrombosis Res 7(6):861; 1975); fibrinogen D fragment (Biokhimiia 43(7):1162; 1978); Thrombosis Res 13(3):443; 1978); Thrombosis Res 13(3):557; 1978); Ukr Biokh Zh 49(3):89; 1977); Ukr Biokh Zh 50(3):357; 1978); fibrinogen fragment E (Nouv Presse Med 7(36):3253; 1978); fibrinopeptide Bbeta (15-42) (Arch Intern Med 1985; 145(6):1033); fibrinogen Bbeta (15-42) (Thromb Res 1982; 25(3):277); fibrinogen-related antigen (Biochem J 1979; 183(3):623); fibrinogen fragment X (Biochim Biophys Acta 1981; 668(1):81); fibrinogen Bbeta (1-42) (Thromb Res 1982; 26(2):111); fibrin fragment E-2 (J Clin Invest 1991; 88(6):2012); fibrinogen peptide 6A (Biochim Biophys Acta 1980; 632(1):87); fibrinopeptide D (Biochemistry 1985; 24(14):3429); fibrinogen fragment Y (Thromb Res 1982; 27(4):377); fibrinopeptide E (Biochemistry 1985; 24(14):3429); fibrinogen peptide 6D (Biochim Biophys Acta 1980; 632(1):87); fibrinogen Bbeta (30-43) (Thomb Res 1985; 37(1):85); Aalpha 1-21 peptide (Ann Intern Med 1987; 107(5):680); fibrinopeptide Bbeta (1-42) (Rinsho Byori 1989; 37(3):234); fibrinogen (gamma 95-264) (J Biol Chem 1981; 256(15):8018); amino-terminal disulfide knot (Thromb Haemost 1984; 51(1):16); fibrin fragment beta (15-118) (Ulu Biokhim Zh 1995 July-August; 67(4):57-64); fibrinogen fragment (Hol-DSK) (Int Arch Allergy Appl Immunol 1983; 70(1):92); fibrinogen Bbeta (1-21) (Thromb Haemost 1984; 51(1):16); des-(Bbeta1-42)-fibrin (J Biol Chem 1990; 265(30):18650); peptide 32 (J Cardiovasc Pharmacol 1994 January; 23(1):103-6); or NDSK-II (Nat Med 2005 March; 11(3):298-304.

GenBank sequence Accession Numbers for each of the chains of fibrinogen are as follows:

| Chain | Gene Name | Nucleic Acid | Protein |
|---|---|---|---|
| Alpha chain | fibrinogen alpha | NM_000508.3 | NP_000499.1 |
| | chain isoform alpha- | | |
| | E preproprotein | | |
| | fibrinogen alpha | NM_021871.2 | NP_068657.1 |
| | chain isoform alpha | | |
| | preproprotein | | |
| Beta chain | fibrinogen beta | NM_005141.4 | NP_005132.2 |
| | chain isoform 1 | | |
| | preproprotein | | |
| | fibrinogen beta | NM_001184741.1 | NP_001171670.1 |
| | chain isoform 2 | | |
| | preproprotein | | |
| Gamma chain | fibrinogen gamma | NM_000509.4 | NP_000500.2 |
| | chain isoform | | |
| | gamma-A precursor | | |
| | fibrinogen gamma | NM_021870.2 | NP_068656.2 |
| | chain isoform | | |
| | gamma-B precursor | | |

Generally speaking, the methods described herein can include obtaining a urine sample from a subject, and determining levels of Fg protein in the sample; alternatively, the methods can include obtaining a kidney biopsy specimen an dimmunostaining it for fibrinogen. Immunostaining for fibrinogen on a kidney biopsy section is done today in some institutions, primarily to classify thrombotic events. The present data (see, e.g., Example 10) indicates that the immunostaining pattern can be used to diagnose AKI and also to differentiate patients who develop AKI following minimal change disease from those who do not.

Any methods known in the art and/or described herein can be used to determine Fg levels in a sample, e.g., immunoassays such as immunoprecipitations, immunofluorescence assays, enzyme immunoassays (EIAs), radioimmunoassays (RIAs), Western blot analysis, enzyme-linked immunosorbent assays (ELISA), antigen capture plates, or chemiluminescence immunoassays (CLIA); enzymatic assays, spectrophotometry, colorimetry, fluorometry, liquid chromatography, gas chromatography, mass spectrometry, Liquid chromatography-mass spectrometry (LC-MS), LC-MS/MS, tandem MS); high pressure liquid chromatography (HPLC), HPLC-MS, and nuclear magnetic resonance spectroscopy. Assay kits are commercially available from AbFrontier Co., Ltd; Abnova Corporation; Alpco Diagnostics; Aniara Corporation; antibodies-online; Cell Sciences; EIAab & USCNLIFE (Wuhan EIAab Science Co., Ltd); GenWay Biotech, Inc.; Innovative Research; and Kamiya Biomedical Company. See also Shibata et al., Nephron. 1995; 69(1):54-8. Where it is desired to measure one or more of the α, β and γ chains of fibrinogen individually, for example, antibodies that detect each of the individual fragments, or mass spectrometry based techniques, can be used to measure the individual peptides quantitatively.

In some embodiments, the level of Fg in the sample is then compared to a reference level, and the presence of a level of Fg in the sample above the reference level indicates that the subject has AKI. A suitable reference level can be determined by one of skill in the art using standard epidemiological and statistical methods; for example, a reference level can be determined based on cohorts of subjects who are selected based on relevant criteria, e.g., subjects determined by other methods to have or to not have AKI. The reference level can be, e.g., a threshold level above which the subject can be diagnosed with AKI (and below which AKI can be ruled out or determined to be less likely). In some embodiments, the reference level is a range, and a level of Fg above the range indicates the presence of AKI, a level below the range indicates the absence of AKI, and a level within the range indicates that the subject is at risk of developing AKI.

In some embodiments, the methods include selecting a subject who is suspected of having, or is at risk of developing AKI. A subject who is at risk of developing AKI is one who has one or more risk factors for AKI as noted above, and/or who has been or is about to be exposed to conditions that are associated with the development with AKI, e.g., a planned procedure that has a risk of renal ischemia/reperfusion injury, e.g., a surgical procedure, imaging study using a contrast agent associated with risk of AKI (e.g., iodinated contrast media) or a subject who has a condition that is associated with an increased risk of AKI, e.g., rhabdomyolysis or abdominal aortic aneurysm, cardiopulmonary bypass, hypoperfusion, or sepsis; or a subject who is about to be or has been treated with drugs that are toxic to the kidney e.g., cisplatin (e.g., for subjects suffering from cancer, e.g., mesotheolima), tenofovir (e.g., for subjects with HIV/AIDS), or gentamicin (e.g., for infections).

In some embodiments, the methods include determining a first level of Fg in a sample from the subject; determining a subsequent level in a sample taken at a later time (e.g., after administration of a treatment, e.g., as known in the art and/or described herein); and comparing the two. No change, or an increase in Fg levels in the subject, indicates that the subject's condition has not improved (e.g., any treatment for AKI was likely not effective), and a decrease in Fg levels indicates that the subject's condition has improved (e.g., any intervening treatment for AKI was effective). In this way a subject's condition can be monitored, e.g., a subject who has AKI or who is at risk of developing AKI. Thus, for example, a subject who is about to undergo a surgical procedure with a risk of AKI can be monitored; a first level of Fg can be determined before the procedure, and subsequent levels of Fg can be determined after the procedure, e.g., at 12, 24, 48, or 72 hours after the procedure. In some embodiments the procedure is a surgical repair of an abdominal aortic aneurysm.

Methods of Treatment

The methods described herein include methods for the treatment of AKI. For example, the methods described herein include the treatment of subjects identified as having AKI, e.g., by a method known in the art or described herein. The methods include administering a therapeutically effective amount of a fibrin derived peptide, e.g., Bβ₁₅₋₄₂ peptide (GHRPLDKKREEAPSLRPAPPPISGGGYR (SEQ ID NO:31)), as described herein, to a subject who is in need of, or who has been determined to be in need of, such treatment.

As used in this context, to “treat” means to ameliorate at least one symptom of AKI. For example, a treatment can result in an improvement in renal function, e.g., a decrease in serum creatinine levels, glomerular filtration rate, BUN, or urine KIM-1 or fibrinogen levels.

Fusion Peptides

In some embodiments, the fibrin derived peptides, e.g., Bβ₁₅₋₄₂ peptides, also include (e.g., are fused in-frame to) a cell-penetrating moiety that facilitates delivery of the peptides to the intracellular space, e.g., HIV-derived TAT peptide, penetratins, transportans, SS peptides (alternating aromatic residues and basic amino acids (aromatic-cationic peptides)), SA, SM, or SNL peptides, or hCT derived cell-penetrating peptides, see, e.g., Caron et al., (2001) Mol Ther. 3(3):310-8; Langel, Cell-Penetrating Peptides: Processes and Applications (CRC Press, Boca Raton Fla. 2002); El-Andaloussi et al., (2005) Curr Pharm Des. 11(28):3597-611; Lindgren et al., Trends Pharmacol Sci. 21(3):99-103 (2000); Zhao et al., J Biol Chem 279:34682-34690 (2004); Szeto, AAPS Journal 2006; 8 (2) Article 32; Deshayes et al., (2005) Cell Mol Life Sci. 62(16):1839-49; Hom et al., J Med. Chem., 46:1799 (2003); Bonny et al., Diabetes, 50:77-82 (2001), and U.S. Pat. Nos. 6,841,535 and 7,576,058 and references cited therein. In some embodiments the cell-penetrating moiety is linked to the peptide, e.g., as a single fusion protein; thus, the invention includes fusion proteins comprising a fibrin derived (e.g., Bβ15-42) peptide as described herein and a cell-penetrating peptide, e.g., TAT, penetratins, transportans, or hCT derived cell-penetrating peptides. In some embodiments, the cell-penetrating peptide is attached to the N-terminus of the fibrin derived (e.g., Bβ15-42) peptide; in some embodiments, the cell-penetrating peptide is attached to the C-terminus of the fibrin derived (e.g., Bβ15-42) peptide. In some embodiments, the fusion protein further comprises a cleavable moiety as known in the art between the cell-penetrating peptide and the fibrin derived (e.g., Bβ15-42) peptide that cleaves off the cell-penetrating peptide, leaving the fibrin derived (e.g., Bβ15-42) peptide intact.

Peptidomimetics

In some embodiments, the peptides disclosed herein can be modified according to the methods known in the art for producing peptidomimetics. See, e.g., Kazmierski, W. M., ed., Peptidomimetics Protocols, Human Press (Totowa N.J. 1998); Goodman et al., eds., Houben-Weyl Methods of Organic Chemistry: Synthesis of Peptides and Peptidomimetics, Thiele Verlag (New York 2003); and Mayo et al., J. Biol. Chem., 278:45746 (2003). In some cases, these modified peptidomimetic versions of the peptides and fragments disclosed herein exhibit enhanced stability in vivo, relative to the non-peptidomimetic peptides.

Methods for creating a peptidomimetic include substituting one or more, e.g., all, of the amino acids in a peptide sequence with D-amino acid enantiomers. Such sequences are referred to herein as “retro” sequences. In another method, the N-terminal to C-terminal order of the amino acid residues is reversed, such that the order of amino acid residues from the N terminus to the C terminus of the original peptide becomes the order of amino acid residues from the C-terminus to the N-terminus in the modified peptidomimetic. Such sequences can be referred to as “inverso” sequences.

Peptidomimetics can be both the retro and inverso versions, i.e., the “retro-inverso” version of a peptide disclosed herein. The new peptidomimetics can be composed of D-amino acids arranged so that the order of amino acid residues from the N-terminus to the C-terminus in the peptidomimetic corresponds to the order of amino acid residues from the C-terminus to the N-terminus in the original peptide.

Other methods for making peptidomimetics include replacing one or more amino acid residues in a peptide with a chemically distinct but recognized functional analog of the amino acid, i.e., an artificial amino acid analog. Artificial amino acid analogs include beta-amino acids, beta-substituted beta-amino acids (“beta3-amino acids”), phosphorous analogs of amino acids, such as b-amino phosphonic acids and b-amino phosphinic acids, and amino acids having non-peptide linkages. Artificial amino acids can be used to create peptidomimetics, such as peptoid oligomers (e.g., peptoid amide or ester analogues), beta-peptides, cyclic peptides, oligourea or oligocarbamate peptides; or heterocyclic ring molecules. Exemplary retro-inverso Bβ15-42 peptidomimetics include RYGGGSIPPPAPRLSPAEERKKDLPRHG (SEQ ID NO:32), wherein the sequences include all D-amino acids.

Modifications

The peptide sequences described herein can be modified, e.g., by modification of one or more amino acid residues of a peptide by chemical means, either with or without an enzyme, e.g., by alkylation, acetylation, acylation, methylation, ADP-ribosylation, ester formation, amide formation, e.g., at the carboxy terminus, or biotinylation, e.g., of the amino terminus. In some embodiments, the peptides are acetylated, e.g., on the free N6 epsilon amino group of Lys7 or Lys8 or on a guanidinium group nitrogen of Arg3, Arg9, Arg 16, or Arg 28). In some embodiments, the peptides are amidated. Methods known in the art can be used to amidate or acetylate the peptides.

In some embodiments, the peptides are modified by the addition of a lipophilic substituent (e.g., a fatty acid) to an amino acid, e.g., to the Lysine. In some embodiments, the peptides include one or more of an N-terminal imidazole group, or a C-terminal amide group. In some embodiments, the epsilon-amino group of Lys34 is substituted with a lipophilic substituent, e.g., of about 4-40 carbon atoms, e.g., 8-25 carbon atoms. Examples include branched and unbranched C6-C20 acyl groups. Exemplary lipophilic substituents, and methods of attaching the same (including via an optional linker) are provided in U.S. Pat. No. 6,268,343 and Knudsen et al., J. Med. Chem. 43:1664-1669 (2000). In some embodiments, the lipophilic substituent is a fatty acid selected from the group consisting of straight-chain or branched fatty acids, e.g., oleic acid, caprylic acid, palmitic acid, and salts thereof.

In some embodiments, the peptide sequences are modified by substituting one or more amino acid residues of the parent peptide with another amino acid residue. In some embodiments, the total number of different amino acids between the sequence-modified peptide and the corresponding native form of the Bβ15-42 peptide is up to five, e.g., up to four amino acid residues, up to three amino acid residues, up to two amino acid residues, or one amino acid residue.

In some embodiments, the total number of different amino acids does not exceed four. In some embodiments, the number of different amino acids is three, two, or one. In order to determine the number of different amino acids, one should compare the amino acid sequence of the sequence-modified Bβ15-42 peptide derivative with the corresponding native Bβ15-42 fragment.

The methods described herein include the manufacture and use of pharmaceutical compositions, which include Bβ15-42 peptide as an active ingredient. Also included are the pharmaceutical compositions themselves.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., a loop diuretic, e.g., furosemide; dopamine agonists, e.g., dopamine or fenoldopam.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

In some embodiments, the methods include administration of an additional therapeutic compound, e.g., a loop diuretic, e.g., furosemide; dopamine agonists, e.g., dopamine or fenoldopam; or administration of renal replacement therapy (RRT). Present options for RRT include hemodialysis (IHD) and peritoneal dialysis (PD), as well as various forms of continuous renal replacement therapy (CRRT) and “hybrid” therapies such as extended duration dialysis (EDD), sustained low-efficiency dialysis (SLED) and the Genius® system. See, e.g., Bagshaw et al., Critical Care Medicine 2008; 36:610-617.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The invention is further described in the following examples, which do not limit the scope of the invention described in the claims.

For examples 1-5, data are expressed as average+standard error. Statistical difference (p<0.05) was calculated by one way ANOVA or student's t-test. P<0.05 was considered significant and represented by ‘*’ where applicable. All graphs were generated by GraphPad Prism (GraphPad, Inc., La Jolla, Calif.). Diagnostic performance (i.e., the ability of a urinary biomarker to identify kidney injury) was assessed by evaluating sensitivity and specificity using the receiver operating characteristic (ROC) curve. The area under the ROC curve (AUC) and 95% confidence interval (CI) were calculated using the non-parametric method 22. The AUC for a diagnostic test ranges from 0.5 (no better than chance alone) to 1.0 (perfect test, equivalent to the gold standard). Statistical analyses were performed with MedCalc for Windows, version 11.5 (Mariakerke, Belgium).

### Example 1

### mRNA Expression of α, β and γ Chains of Fibrinogen (Fg) Increases in the Kidney Following Ischemia Reperfusion Injury

To identify early genes modulating kidney injury and repair process, gene expression profiling was conducted in the cortex and medulla of rat kidney following 20 minutes bilateral renal ischemia/reperfusion (I/R).

Male Wistar rats (280-320 g) and male C56Bl/6 mice (22-25 g) were purchased from Harlan Laboratories (Indianapolis, Ind.) and Charles River Laboratories (Wilmington, Mass.) respectively. The animals were maintained in central animal facility over wood chips free of any known chemical contaminants under conditions of 21±1° C. and 50-80% relative humidity at all times in an alternating 12 h light-dark cycle Animals were fed with commercial rodent chow (Teklad rodent diet #7012), given water ad lib, and were acclimated for 1-week prior to use.

For whole genome expression profiling studies, nine male Wistar rats underwent ischemia reperfusion (I/R) surgery and three rats underwent sham surgery simulating I/R. In order to perform I/R surgery, the rats were anesthetized using pentobarbital sodium (30 mg/kg, ip) and renal ischemia was induced by nontraumatic vascular clamps over the pedicles for 20 min as described before (Vaidya et al., Kidney Int. 2009; 76(1):108-114; Vaidya et al., Nat Biotechnol. 2010; 28(5):478-485). Upon release of the clamps, the incision was closed in two layers with 2-0 sutures. The sham rats underwent anesthesia and a laparotomy only and were sacrificed after 24 h. The rats in I/R group were further divided in subgroups of three rats each and sacrificed after 6, 24, and 120 h of reperfusion. To confirm the results of gene expression analysis twenty male Wistar rats underwent 20 minutes bilateral I/R surgery and five rats underwent sham surgery as described above and were sacrificed at 6, 24, 72 and 120 h following reperfusion (n=5/timepoint).

For genome-wide expression analysis, RatRef-12 bead array (Illumina, San Diego, Calif.) was used which contains about 22,523 50-mer oligonucleotide probes primarily based on NCBI RefSeq database (Release 16). Gene expression and hybridization array dataset has been submitted to the NCBI Gene expression omnibus. Accession: GSE27274. Total RNA was extracted from 30 mg of frozen tissue samples using TRIZOL reagent (Invitrogen, Carlsbad, Calif.) according to manufacturer's instructions. Integrity of the isolated total RNA was determined by 1% agarose gel electrophoresis and the RNA concentration was measured by ultraviolet light absorbance at 260 nm using the Nanodrop 2000C spectrophotometer (Thermo Scientific, Rockford, Ill.). Aliquots of RNA were converted into ds-cRNA and biotinylated using the Illumina TotalPrep RNA Amplification Kit (Ambion, Austin, Tex., USA). The cRNA samples were then labeled with streptividin-Cy3 and hybridized onto RatRef-12 Expression Beadchip. The image was scanned using the Illumina BeadArray Reader and the data was analyzed by Illumina Beadstudio software (version 3.3.7). For non-redundant 22,523 symbols, the intensity profiles were quantile normalized. Median absolute deviation (MAD) was used to select highly variable genes (MAD>0.4; n=1571) for subsequent analysis. Hierarchical clustering was performed using 1-Pearson correlation coefficient as distance with average linkage option.

Blood and urine analysis was performed as follows. At sacrifice, blood was collected from dorsal aorta in heparinized tubes. Serum creatinine (SCr) concentrations were measured using a Beckman Creatinine Analyzer II. Blood urea nitrogen (BUN) was measured spectrophotometrically at 340 nm using a commercially available kit (Thermo Scientific, Rockford, Ill.) as described before (Krishnamoorthy et al., Toxicol Sci. 2010; 118(1):298-306).

Urines were collected by placing animals in individual metabolic cages and one ml of RNAlater (Ambion, Austin, Tex.) was added to the tubes to preserve RNA. Urinary Kidney Injury Molecule-1 (Kim-1 in rats and KIM-1 in humans) was measured using previously established luminex-based assays (Vaidya et al., Nat Biotechnol. 2010; 28(5):478-485; Vaidya et al., Clinical and Translational Science. 2008; 1(3):200-208). Urinary Kim-1 in mice was measured using a luminex-based assay. Urinary N-acetyl-β-_{D}-glucosaminidase (NAG) was measured spectrophotometrically according to the manufacturers' protocols (Roche diagnostics, Basel Switzerland). Urinary creatinine concentration was used to normalize biomarker measurements in order to account for the influence of urinary dilution on biomarker concentrations. Fibrinogen in mouse, rat and human urines was measured using a commercially available species-specific luminex assay based kit from Millipore (Billerica, Mass.).

This reversible model of kidney injury results in elevated kidney dysfunction (measured by serum creatinine (SCr) and blood urea nitrogen (BUN)) (FIG. 1A) and proximal tubular injury (measured by kidney injury molecule-1 (Kim-1) mRNA levels and histopathological findings characterized by proximal tubular necrosis and apoptosis) (FIG. 1C) at 24 h of reperfusion followed by recovery at 120 h. Highly variable genes (median absolute deviation >0.4; n=1571) were selected and hierarchical clustering was performed to investigate their co-expression pattern during kidney regeneration after ischemic injury (FIG. 1A). The selected genes include the previously identified candidate genes lipocalin-2 (LCN2) (Mishra et al., J Am Soc Nephrol. 2003; 14(10):2534-2543), clusterin (CLU) (Dieterle, Nat Biotechnol. 2010; 28(5):463-469), tissue inhibitor of metalloproteinase-1 (TIMP1) (Amin et al., Environ Health Perspect. 2004; 112(4):465-479), and kidney injury molecule-1 (Kim-1) (Vaidya et al., Nat Biotechnol. 2010; 28(5):478-485). While the up regulation of LCN2 and CLU were more dominant in renal medulla compared to cortex, TIMP1 and Kim-1 were up regulated both in cortex and medulla at 24 hr after ischemic injury. There was a local cluster of genes that included Fgβ and Fgγ chains whose expression pattern was similar to Kim-1, i.e., clear up-regulation after 24 hr of ischemic injury both in cortex and medulla. The probe for Fgα chain was absent in the RatRef-12 chip from Illumina.

Therefore, the expression profiles of Fgα, Fgβ and Fgγ chains were further evaluated by real-time PCR (RT-PCR) as follows. The isolated RNA was treated with Quantitect Reverse Transcription kit (Qiagen Sciences, Germantown, Md.). Real Time PCR of the tissue samples was performed with Quantifast SYBR Green (Qiagen Sciences, Germantown, Md.) using a CFX96 RT-PCR instrument (Biorad, Hercules, Calif.) (Krishnamoorthy et al., Toxicol Sci. 2010; 118(1):298-306). Primers were designed to amplify 120-150 base pair fragment with the following cycle conditions: 95° C. for 3 min, the following steps were repeated 40 times: 95° C. for 30 sec, 55° C. for 30 sec. Forward and reverse primer sequences for rat and mouse specific genes were designed using MacVector software (MacVector Inc., Cary, N.C.) and are listed in Table 1.

Expression levels were evaluated in kidney (cortex and medulla) (FIG. 1A), liver (FIG. 1A), lung, spleen and heart (FIG. 1D) over time; there was a modest elevation (3-fold) of Fgα and Fgγ chains in the liver at early time points (FIG. 1A), but no significant change over time in the mRNA levels of any of the chains in lung, spleen and heart (FIG. 1D). The medulla showed significantly higher expression of all three chains as compared to cortex with the highest up regulation after 72 h of reperfusion (Fgα chain-14 fold, Fgβ chain-50 fold and Fgγ chain-10 fold) (FIG. 1A).

TABLE 1
Real Time-PCR primers used for
the quantification of mRNA expression levels

| | | | SEQ |
|---|---|---|---|
| | | | ID |
| Gene | F/R | Sequence | NO: |
| CD68 | F | TCTTTCTCCAGCTGTTCACC | 1 |
| | R | ATGATGAGAGGCAGCAAGAG | 2 |
| Gapdh | F | TCCGCCCCTTCTGCCGATG | 3 |
| | R | CACGGAAGGCCATGCCAGTGA | 4 |
| ICAM1 | F | TGTTTTGCTCCCTGGAAGGC | 5 |
| | R | AGTCACTGCTGTTTGTGCTCTCC | 6 |
| IL-1β | F | ACCTGTCCTGTGTAATGAAAGACG | 7 |
| | R | TGGGTATTGCTTGGGATCC | 8 |
| IL-6 | F | CAAGAGACTTCCATCCAGTTGCC | 9 |
| | R | CATTTCCACGATTTCCCAGAGAAC | 10 |
| IL-10 | F | CAGCCTTGCAGAAAAGAGAG | 11 |
| | R | GGAAGTGGGTGCAGTTATTG | 12 |
| Mouse Fgα chain | F | TGTGGAGAGACATCAGAGTCAATG | 13 |
| | R | CGTCAATCAACCCTTTCATCC | 14 |
| Mouse Fgβ chain | F | CTATGGCTGCTGCTGCTATTG | 15 |
| | R | GGCTCTTCCTTTCTCCTGTCAAC | 16 |
| Mouse Fgγ chain | F | TGTGGCTACCAGAGATAACTGTTG | 17 |
| | R | ATGTCTTCCAGCGTTCGGAG | 18 |
| Mouse Kim-1 | F | GGAGATACCTGGAGTAATCACACTG | 19 |
| | R | TAGCCACGGTGCTCACAAGG | 20 |
| Rat Fgα chain | F | AGCAGCCCAGCGACAAGAAAAG | 21 |
| | R | GCAGTCAGAACCATCATCCGAAG | 22 |
| Rat Fgβ chain | F | TAGCCTAAGACCTGCCCCTC | 23 |
| | R | ACCGTGAACACAGCCTCCTG | 24 |
| Rat Fgγ chain | F | GGACAATGACAACGACAAGTTCG | 25 |
| | R | ATACCAGCGGGTTTTCCAGG | 26 |
| Rat Kim-1 | F | TATTTGGGGGAACAGGTTGC | 27 |
| | R | CAAGTCACTCTGGTTAGCCGTG | 28 |
| TNFα | F | AGAAGAGGCACTCCCCCAAAAG | 29 |
| | R | TTCAGTAGACAGAAGAGCGTGGTG | 30 |

### Example 2

### Immunoreactivity of Fgα, Fgβ and Fgγ Chains in the Kidney

Immunostaining, to evaluate the cellular expression profile of Fg whole protein and Fgα, Fgβ and Fgγ chains was performed as follows. Kidney tissues were perfused with cold PBS before harvesting and then fixed in formalin for 16 h and embedded in paraffin. The sections incubated overnight at 4° C. in rabbit monoclonal anti-Fibrinogen alpha (Epitomics, Burlingame, Calif.), rabbit polyclonal anti-Fibrinogen beta (ProteinTech Group, Chicago, Ill.), rabbit polyclonal anti-Fibrinogen gamma (ProteinTech Group, Chicago, Ill.), anti-rat fibrinogen (Nordic Immunological Laboratories, Tilburg, The Netherlands), anti-human fibrinogen (Sigma-Aldrich, St. Louis, Mo.) and rabbit monoclonal anti-Ki67 (Vector Laboratories, Burlingame, Calif.). The primary antibody was detected using goat anti-rabbit Cy3 labeled and donkey anti goat Cy3 labeled secondary antibodies (Jackson ImmunoResearch Laboratories, West Grove, Pa.). DAPI (Sigma Aldrich, St. Louis, Mo.) was used for nuclear staining. The tissue sections were mounted using ProLong Gold Antifade Reagent (Invitrogen, Carlsbad, Calif.). The images were captured at 100× for rats, 60× for mice and humans using NIKON Eclipse 90i fluorescence microscope.

The results revealed immunoreactive protein for all three chains and Fg whole molecule in renal tubular cells. Positive staining was observed using the anti-Fgβ chain and anti-Fg whole molecule antibodies in the interstitial spaces, indicative of extracellular Fg. In sham kidneys, the Fga chain was expressed with fine granular cytoplasmic reactivity and more pronounced expression at the peak of injury by 24 h. Fgα chain immunoreactive protein, as detected by the monoclonal antibody, continued to be expressed in the proximal tubular epithelial cells and in the glomeruli throughout the time course of injury. Fgβ chain immunoreactive molecules in the uninjured kidneys, assessed by a polyclonal antibody against Fgβ chain, showed focal reactivity in the renal interstitium. At the peak of injury by 24 h, Fgβ immunoreactivity distinctly outlined the peritubular capillaries and a small proportion of tubular epithelial cells expressed the Fgβ chain immunoreactive component in their cytoplasm. By 72 h, intense, irregular and coarse distal tubular staining and a distinct luminal outline along proximal tubules featured. The Fgγ chain staining, assessed by a polyclonal antibody against Fgγ chain, was primarily located in the distal tubules and collecting ducts with a diffuse cytoplasmic distribution that gravitated along the basolateral side in uninjured kidneys. At the peak of injury by 24 h, the Fgγ chain immunoreactive protein stained in a coarse granular pattern, distributed centrally in the cytoplasm in the distal tubules and collecting ducts. By 24 h, the Fgγ chain in the cortex was confined towards the apical side of the proximal tubules while in the medulla, the cellular debris of injured S3 segments non-specifically stained for the Fgγ chain as well. By 72 h, Fgγ chain immunoreactive proteins showed a mixed pattern that resembled sham and 24 h injured kidneys in the staining and distribution patterns. The expression of fibrinogen whole molecule was identified in a linear pattern along the apical surface of epithelial cells as well as along the glomerular basement.

A consistent pattern of increased expression of Fg and its chains was observed in human kidney biopsy sections obtained from patients pathologically diagnosed with acute tubular injury (ATI) as compared to patient without evidence of ATI. Human kidney biopsy sections were classified as patient without evidence of acute tubular injury (ATI): 58 year old female diagnosed with renal oncocytoma and patient with ATI: 78 year old male diagnosed with active glomerulonephritis with diffuse proliferative and cresentric pattern of injury, active interstitial nephritis, active tubular injury involving focal tubular atrophy and interstitial fibrosis (30%). All three chains along with Fg were present in the interstitium in both ATI and non-ATI patients, in addition to which Fgγ chain and Fg were predominantly expressed on the apical side of the tubules in the ATI patient.

### Example 3

### Increased Urinary Levels of Fg in Rats and Humans Serve as a Potential Biomarker for Acute Kidney Injury

It was hypothesized that if Fg was secreted into the urine upon injury, then urinary Fg may serve as a biomarker for kidney injury. Following 20 min bilateral renal I/R injury in rats, an approximately 100-fold increase in urinary Fg concentration was observed as early as 6 h (FIG. 2A, top) that remained higher than baseline till day 5 (˜4 fold) following reperfusion, correlating with proximal tubular necrosis as assessed by histopathologic injury and elevated urinary N-acetyl-β-D-glucosaminidase (NAG) (FIG. 2A, middle) as well as urinary kidney injury molecule-1 (Kim-1) (FIG. 2A, bottom). There was no increase in plasma Fg (FIG. 2D) levels after sham or kidney I/R injury as compared to rats that did not undergo sham or I/R surgery.

To evaluate the performance of urinary Fg in distinguishing healthy volunteers against patients with chronic kidney disease (CKD) and/or acute kidney injury (AM), urinary Fg was measured in 25 patients admitted to the intensive care unit with abnormal serum creatinine (≧1.5 mg/dL) with established kidney damage from a variety of causes and 25 healthy volunteers using a commercially available species-specific LUMINEX assay based kit from Millipore (Billerica, Mass.). Critically ill patients in the intensive care unit with elevated SCr>1.5 mg/dL were recruited. Causes of acute kidney injury (AKI) or chronic kidney disease (CKD) were obtained by detailed chart review including the treating nephrologist's consultation note and evaluation of laboratory data by a co-author not involved in the patients' care (SSW). Healthy volunteers were recruited from the staff at BWH. Healthy volunteers were excluded if they reported a recent hospitalization, diagnosis of chronic kidney disease, or treatment with nephrotoxic medications (non-steroidal anti-inflammatory drugs were allowed). Urinary Fg was also compared against two other well-studied AKI or CKD biomarkers, NAG and KIM-1. Demographic and clinical data are shown in Table 2.

TABLE 2
Demographic and clinical characteristics of human subjects

| | Acute kidney injury (AKI) | |
|---|---|---|
| | or Chronic kidney | Healthy |
| | disease (CKD) | volunteers** |
| | (N = 25) | (N = 25) |
| Mean age*, years, ± | 64.8 ± 19.5 | 35.6 ± 10.7 |
| SD | | |
| Female^{†} | 64% | 68% |
| Black^{‡} | 20% | 16% |
| Cause of elevated | AKI from shock or sepsis | — |
| serum creatinine (SCr) | (72%), obstruction (4%), | |
| | multifactorial (12%), pre-renal | |
| | (4%), CKD (8%) | |
| Mean (SD) peak SCr | 4.5 (4.7) mg/dL | — |

*P < 0.001
^{†}P = 0.73
^{‡}P = 0.68
**Healthy volunteers were excluded if they reported a diagnosis of chronic kidney disease; serum creatinine was not measured.

Median urinary concentration of Fg was significantly higher in patients with AKI and CKD than in healthy volunteers (p<0.001) (FIG. 2B) and corresponded with the increased levels of urinary NAG and KIM-1 (FIG. 2E). The diagnostic ability of urinary Fg to distinguish between patients with AKI or CKD versus patients without kidney injury was 0.98 as calculated by area under the receiver operating characteristic curve (ROC) (FIG. 2C).

### Example 4

### Fibrinogen Bβ₁₅₋₄₂ Protects the Kidney Against Ischemia-Reperfusion (I/R) Injury

Given that in the present model Fgβ chain was the highest up regulated gene following kidney injury amongst the three chains (FIG. 1A) and the fact that exogenous Bβ₁₅₋₄₂ peptide administration has been shown to protect against myocardial I/R injury (Hallen et al., EuroIntervention;5(8):946-952; Petzelbauer et al., Nat Med. 2005; 11(3):298-304; and Atar et al., J Am Coll Cardiol. 2009; 53(8):720-729) and lung injury (Matt et al., Am J Respir Crit Care Med. 2009; 180(12):1208-1217), the therapeutic potential of Bβ₁₅₋₄₂ peptide in renal I/R injury was evaluated.

Endotoxin free Bβ₁₅₋₄₂ (GHRPLDKKREEAPSLRPAPPPISGGGYR) and random peptide (DRGAPAHRPPRGPISGRSTPEKEKLLPG) were custom synthesized (Invitrogen, Carlsbad, Calif.) with 95% modification and N-terminal amine group addition and free acid modification. Bβ₁₅₋₄₂ or random peptide (3.6 mg/kg) was administered intravenously (iv) 1 min after reperfusion following 27 min bilateral renal I/R injury in C57BL/6 mice (n=5 to 10/group). Forty male C57Bl6 wild type mice were anesthetized using pentobarbital sodium (30 mg/kg, ip) and subjected to 27 min of bilateral renal I/R surgery by the retroperitoneal approach. Sham surgery was performed with exposure of both kidneys but without induction of ischemia. Immediately upon the start of reperfusion, 3.6 mg/kg of Bβ15-42 or random peptide were administered intravenously to the mice via tail vein. One ml of warm saline (37° C.) was injected ip three hours after surgery for volume supplementation. Mice (n=5-10/group) in the respective groups (sham or I/R administered Bβ15-42 or random peptide) were sacrificed at 24 and 48 h following reperfusion using overdose of pentobarbital (180 mg/kg, ip).

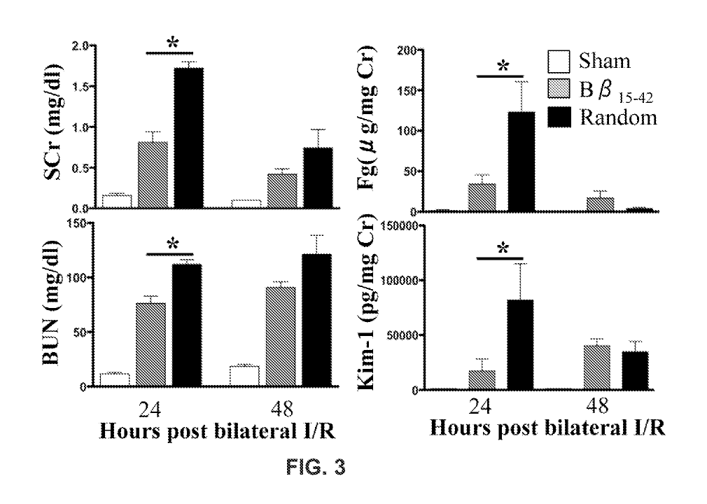

A significant reduction in the infarct size and vascular congestion (outlined by white dots) was observed. Approximately 50% reduction in kidney dysfunction [measured by serum creatinine (SCr), blood urea nitrogen (BUN)] and kidney proximal tubular injury (measured by urinary levels of kidney injury molecule-1 and Fg) (FIG. 3), and a significant decrease in proximal tubular damage in the outer stripe of outer medulla (histopathological evaluation of H & E stained kidney sections) was recorded at 24 h after I/R injury in the mice administered Bβ₁₅₋₄₂ peptide as compared to random peptide. The kidney injury and dysfunction parameters appeared to decrease by 48 h suggesting the onset of a complete structural and functional recovery in both groups.

### Example 5

### Decreased Apoptosis and Increased Tissue Repair in the Kidneys Bβ₁₅₋₄₂ Treated Mice as Compared to Mice Treated with Random Peptide Following Ischemia-Reperfusion (I/R) Injury

To elucidate the mechanism of Bβ₁₅₋₄₂-induced protection in I/R mice, candidate markers of inflammation, leukocyte infiltration, apoptosis and proliferation were measured in kidney tissues over time. There was no difference in mRNA levels of inflammatory cytokines (IL-1β, IL-6, IL-10, TNFα, ICAM), or macrophage marker (CD68) between the Bβ₁₅₋₄₂ and random peptide treatment groups (FIG. 4A). Similarly leukocyte infiltration (measured by myeloperoxidase staining) also appeared to be similar between the two groups (FIG. 4B).

Apoptosis was measured in kidney tissues by TUNEL assay using the In Situ Cell Death detection kit (Roche Applied Science, Indianapolis, Ind.) according to manufacturer's instructions (Krishnamoorthy et al., Toxicol Sci. 2010; 118(1):298-306). The number of TUNEL positive apoptotic cells in the renal medulla was similar at 24 h. However, at 48 h there was a significant decrease in apoptosis (p<0.05) in the mice administered Bβ₁₅₋₄₂ as compared to random peptide. Interestingly, a significant number of cells appeared to be in a proliferative state (Ki67 positive) in the renal medulla at 48 h following administration of the Bβ₁₅₋₄₂ peptide as compared to random peptide administration.

In vitro experiments using proximal tubular epithelial cells (LLC-PK1) were also performed. The renal tubular epithelial cell line, LLC-PK1, established from pig renal cortex was obtained from ATCC (Manassas, Va.) and maintained in DMEM containing 10% FBS. Two thousand five hundred LLC-PK1 cells were plated in 96 well plate for 24 h in DMEM 10% fetal bovine serum (FBS) at which time they formed a 50% confluent monolayer in the well. They were pretreated with 6 μM of Bβ₁₅₋₄₂ or random peptide for 6 h and were immersed in 100 μl of mineral oil on top of DMEM medium without any serum for 6 h. This oil immersion simulates in vivo ischemic conditions by restricting cellular exposure to oxygen and nutrients as well as limiting metabolite washout (Meldrum et al., J Surg Res. 2001; 99(2):288-293). After 6 h, the mineral oil was removed and cells were incubated with 6 μM of Bβ₁₅₋₄₂ or random peptide for 48 h in serum free conditions. Bromodeoxyuridine (5-bromo-2-deoxyuridine (BrdU) was measured as an index of cell proliferation by incubating cells with BrdU for 2 h before harvesting and the absorbance was quantified using a spectrophotometer at 450 nm wavelength as per manufacturer's instructions (Millipore, Bellerica, Mass.). Absorbance obtained from untreated cells was taken as 100% (n=6 wells/group) and the experiment was repeated twice.

The results mimicked the in vivo findings in demonstrating a protective effect of Bβ₁₅₋₄₂ from hypoxic injury by stimulating renal epithelial cell proliferation (FIG. 4E) suggesting that the Bβ₁₅₋₄₂ peptide promotes an efficient resolution of ischemic injury by inducing rapid tissue regeneration response, thereby decreasing the necrosis and apoptosis in the kidney.

### Example 6

### Kidney Ischemia/Reperfusion Injury in Rats Results in Significant Upregulation and Excretion of Fibrinogen Correlating with Histopathological Injury

The present example evaluated the diagnostic performance of urinary Fg following 30 minutes bilateral renal ischemia/reperfusion-induced reversible injury in rats.

Male Wistar rats (280-320 g) were subjected to bilateral renal ischemia reperfusion (PR) by clamping both renal arteries under anesthesia (30 mg/kg pentobarbital, ip) for 30 min as previous described (Vaidya et al., Nat Biotechnol 2010; 28: 478-485). Rats were euthanized by an overdose of phenobarbital (180 mg/kg) and were sacrificed at 3, 6, 12, 18, 24, 72, 120 and 168 h following reperfusion (n=4). Sham rats underwent anesthesia and a laparotomy only and were sacrificed after 24 h and used as controls.

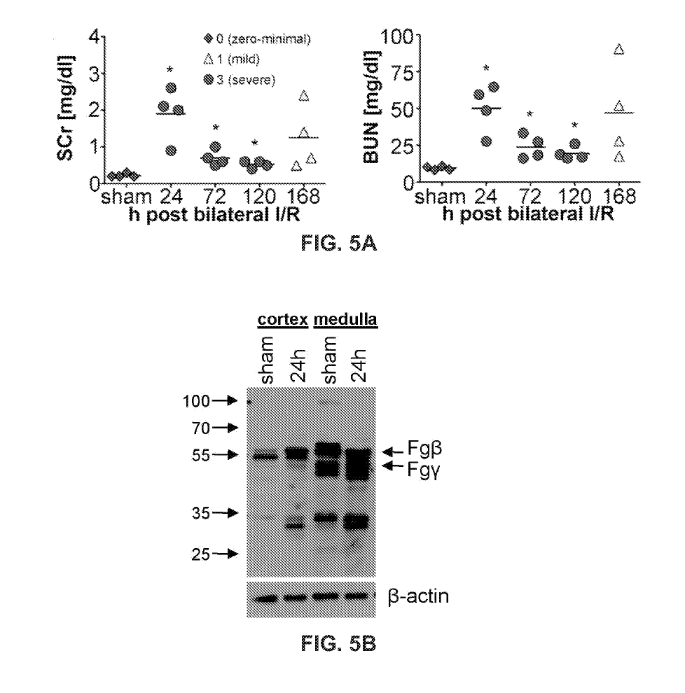

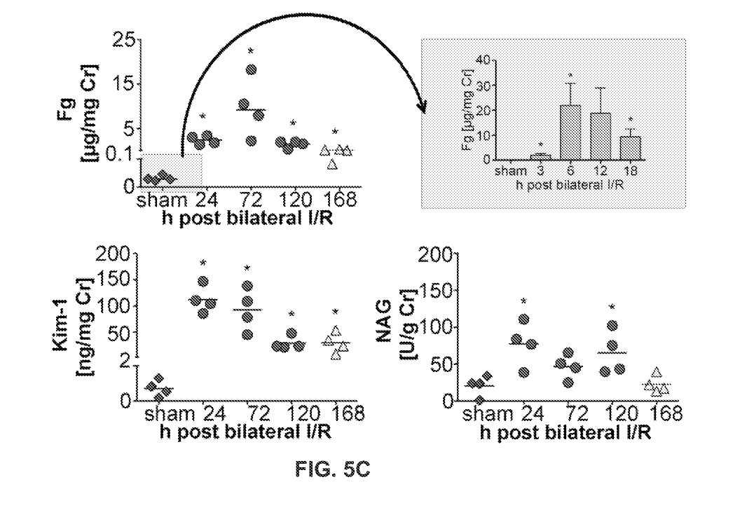

Thirty min of bilateral renal ischemia following reperfusion resulted in peak of kidney injury and dysfunction at 24 h as measured by increase in serum creatinine (SCr), blood urea nitrogen (BUN), and histopathological damage (FIG. 5A). Serum creatinine (SCr) was measured using a Beckman Creatinine Analyzer II and urine creatinine (uCr) was measured using the Creatinine Assay Kit (Cayman, Ann Arbor, Mich.) according to the manufacturers' protocols. Blood urea nitrogen (BUN) was measured spectrophotometrically at 340 nm using a commercially available kit (Thermo Scientific, Rockford, Ill.) as previously described (Krishnamoorthy et al., Toxicol Sci 2010; 118: 298-306).

Kidney parenchyma revealed extensive tubulointerstitial damage at 24 h, particularly prominent at the corticomedullary junction, with marked injury of the S3 segments of the proximal tubules. The individual tubules showed distension of their lumens and extensive degenerative changes of the epithelial cells, with widespread necrosis and collections of cellular debris within the tubule lumens. At 120 h and 168 h following reperfusion, kidneys showed mild tubular distension and prominent reactive changes in the epithelial cell layer. Occasional mitotic figures were detected and the nuclei were enlarged and revealed prominent nucleoli.

RNA extraction and qRT-PCR was performed as follows. At necropsy, tissue was collected, sliced into small fragments and flash frozen in liquid nitrogen and stored at −80° C. freezer. Kidneys from the rats were separated into medulla and cortex. Total RNA was isolated from tissue using the Trizol-chloroform method as described before (Krishnamoorthy et al., Blood 2011; 118: 1934-1942; Krishnamoorthy et al., Toxicol Sci 2010; 118: 298-306). The concentration of total isolated RNA was measured at 260 nm using a NanoDrop spectrophotometer (Thermo Fisher) and integrity was determined by 1% agarose gel electrophoresis. 1 μg of RNA was reverse transcripted into cDNA using QuantiTect® Reverse Transcription Kit from Qiagen according manufactures' instructions. The expression profiles of Fgα, Fgβ, Fgγ and Kim-1 were evaluated with quantitative real-time PCR using QuantiFast SYBR Green PCR Kit (Qiagen) on a CFX96 RTPCR instrument (Biorad). Amplification was carried out using the following temperature profile: 3 min enzyme activation at 95° C. followed by 40 cycles of 95° C. for 10 s, and 55° C. for 30 s. GAPDH was used as reference gene and primer sequences were as previously described (Krishnamoorthy et al., Blood 2011; 118: 1934-1942).

Significantly elevated mRNA expression of all three chains Fgα, Fgβ and Fgγ was observed following I/R in the kidney (Table 3). Among the three chains Fgβ showed the highest expression after 72 h of reperfusion with an ˜26-fold increase in cortex and 75-fold in medulla whereas Fga showed only a ˜5-fold increase in both, medulla and cortex (Table 3). The mRNA up-regulation of Fgγ was slightly higher in cortex compared to medulla.

TABLE 3
mRNA expression of Fgα, Fgβ, and Fgγ significantly increases following
ischemia/reperfusion or cisplatin-induced kidney tubular injury¹.

| | mRNA expression (fold change relative to sham/0h) | | | |
|---|---|---|---|---|
| | Fgα | Fgβ | Fgγ | Kim-1 |
| (a) Ischemia/ | | | | |
| Reperfusion | | | | |
| [h post I/R] | | | | |
| cortex: sham | 1.0 ± 0.1 | 1.0 ± 0.1 | 1.0 ± 0.2 | 1.0 ± 0.3 |
| 24 | 3.0 ± 0.6 | 22.3 ± 7.6 | 17.1 ± 4.0 | 364.7 ± 32.8 |
| 72 | 2.9 ± 0.4 | 26.2 ± 5.9 | 8.2 ± 1.1 | 208.9 ± 55.1 |
| 120 | 4.4 ± 1.0 | 23.1 ± 7.8 | 13.6 ± 2.2 | 125.8 ± 35.4 |
| 168 | 4.6 ± 0.1 | 19.3 ± 4.0 | 8.2 ± 1.3 | 108.5 ± 41.2 |
| medulla: sham | 1.0 ± 0.2 | 1.0 ± 0.2 | 1.0 ± 0.2 | 1.0 ± 0.4 |
| 24 | 3.8 ± 0.8 | 40.2 ± 8.7 | 4.8 ± 1.6 | 325.9 ± 95.7 |
| 72 | 6.1 ± 1.8 | 75.6 ± 24.8 | 9.6 ± 2.4 | 318.2 ± 144.3 |
| 120 | 5.0 ± 2.1 | 46.8 ± 23.4 | 9.6 ± 3.9 | 159.1 ± 67.6 |
| 168 | 2.9 ± 1.0 | 27.6 ± 11.1 | 2.8 ± 0.9 | 76.7 ± 40.7 |
| (b) Cisplatin | | | | |
| [h post 20 mg/kg] | | | | |
| 0 | 1.0 ± 0.0 | 1.0 ± 0.5 | 1.0 ± 0.2 | 1.0 ± 0.1 |
| 24 | 9.6 ± 2.5 | 1.8 ± 0.3 | 25.0 ± 7.8 | 5.0 ± 1.0 |
| 48 | 27.8 ± 7.0 | 17.3 ± 6.8 | 183.2 ± 46.6 | 18.1 ± 4.8 |
| 72 | 25.3 ± 2.9 | 23.0 ± 7.6 | 242.4 ± 32.3 | 23.9 ± 2.7 |

Immunoblotting was carried out as follows. Total protein was isolated by homogenization of kidney tissues in Ripa-buffer containing complete Protease Inhibitor Cocktail tablets (Roche Applied Science, Indianapolis, N). Protein concentration was determined using the BCA Protein Assay Kit (Pierce, Rockford, Ill.) according to manufacturer's instructions. Equal amount of protein was loaded on 12% polyacrylamide gel. The proteins were separated by SDS-PAGE and transferred to a nitrocellulose membrane. After blocking in 5% Blotting Grade Blocker non-fat dry milk (Biorad, Hercules, Calif.) for 1 h at room temperature the membranes were incubated with the polyclonal anti-fibrinogen antibody (1:1000 dilution, Nordic Immunology, Eindhoven, NL) over night at 4° C. After 3 washings steps with TBST the blots were incubated in horseradish peroxidase conjugated secondary antibody (GE healthcare, Buckinghamshire, UK) and antigens on the blots were revealed using enhanced chemiluminescence kit (GE healthcare, Buckinghamshire, UK) by autoradiography. A monoclonal anti-β-actin antibody (Sigma, St. Louis, Mo.) was used as loading control.

Immunoreactivity of Fibrinogen (Fg) and fibrin derived peptides was significantly increased in cortex and medulla at 24 h following ischemia/reperfusion injury as compared to sham surgery (FIG. 5B). Urinary Fg increased significantly after 24 h (˜80-fold) following reperfusion, peaked at 72 h (˜315-fold) and returned back by 120 h (˜50-fold) (FIG. 5C). Urinary excretion of advanced kidney injury biomarkers, kidney injury molecule-1 (Kim-1) and N-acetyl-β-D-glucosaminidase (NAG), also showed highest levels after 24 h compared to sham surgery (FIG. 5C).

The early diagnostic capability of urinary Fg was assessed using urine samples collected at 3, 6, 12 and 18 h following 30 min bilateral renal I/R injury. Urine analyses were performed as follows. Urinary Kidney Injury Molecule-1 (Kim-1 in rats and KIM-1 in humans) was measured by using established luminex-based assays (Vaidya et al., Annu Rev Pharmacol Toxicol 2008; 48: 463-493; Vaidya et al., Nat Biotechnol 2010; 28: 478-485). Levels of Kim-1 in mice urine were determined using a recently established luminex-based assay in the Bonventre laboratory. Urinary N-acetyl-β-D-glucosaminidase (NAG) was measured spectrophotometrically according to manufacturers' instructions (Roche Diagnostics, Basel, Switzerland). Fibrinogen protein in urine of humans, rats and mice was measured using commercially available species-specific luminex based assay kits from Millipore (Billieria, Mass.) (Krishnamoorthy et al., Blood 2011; 118: 1934-1942).

The results are shown in the boxed inset to FIG. 5C). Urinary Fg excretion was increased as early as 3 h (˜60 fold) further escalating at 6 h (˜700 fold).

### Example 7

### Cisplatin-Induced Nephrotoxicity in Mice Results in Marked Increase in Fibrinogen Corresponding to Histological Damage

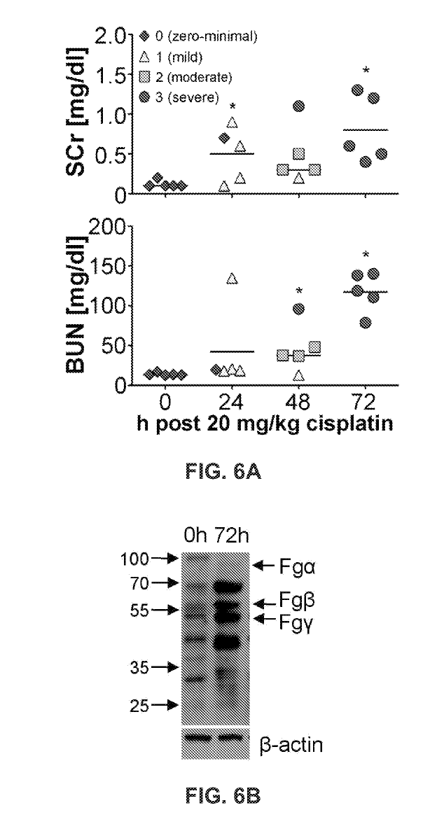

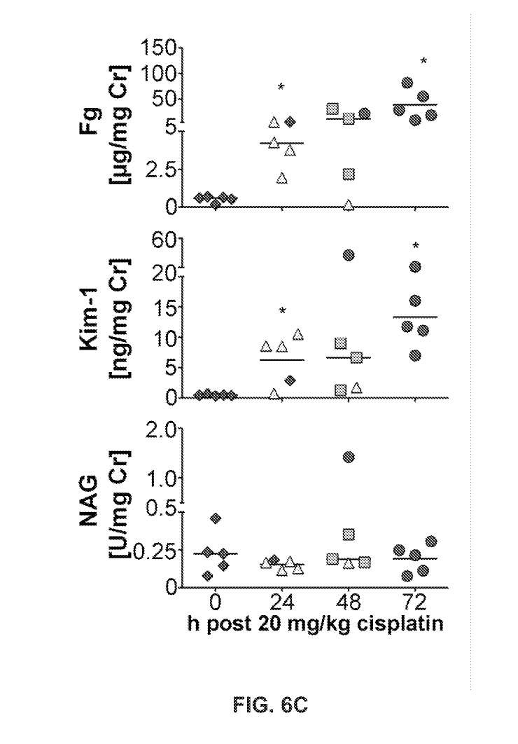

In an irreversible model of kidney injury in mice induced by a single injection of 20 mg/kg cisplatin an increase in SCr and BUN indicative of impaired kidney function was observed at 24-48 h and peaked at 72 h (FIG. 6A). Cisplatin-induced tubulointerstitial damage was most prominent in the superficial renal cortex. The initial injury was manifested by mild tubular distension and a low epithelial lining in most tubules; some tubules revealed vacuolization of their cytoplasm. At 48 hours, injured tubules also revealed single cell death in some tubules, with karyopyknosis and accumulation of cellular debris in few tubular lumens. Following 72 hours of cisplatin administration, widespread epithelial cell necrosis, with sloughing of the epithelium, denuded tubular basement membranes, and necrotic debris filling the lumens of many tubules were seen in the kidneys.

Increased mRNA expression of Fgα, Fgβ and Fgγ in kidneys of cisplatin treated mice was detected as early as 24 h (Table 3). In contrast to I/R injury, where Fgβ showed the highest elevation amongst all three chains in the kidney, administration of cisplatin led to a massive increase in Fgγ (˜250-fold after 72 h) compared to Fgα (˜25-fold) and Fgβ (˜23-fold) (FIG. 6B). A similar increase in fibrinogen protein expression in the kidney was observed at 72 h following cisplatin administration. Approximately 70-fold higher levels of urinary Fg were detected as early as 24 h following cisplatin administration corresponding with ˜30-fold increase in urinary Kim-1 concentration while urinary excretion of NAG remained unchanged (FIG. 6C).

### Example 8

### Specificity of Urinary Fibrinogen as Biomarker of Kidney Injury

To evaluate the specificity of fibrinogen as an AKI biomarker, rats were treated with a well-established hepatotoxicant galactosamine (1.1 mg/kg, ip). After 24 h of galactosamine administration the liver showed extensive hepatocellular damage with a significant increase in the activity of the liver enzymes alanine aminotransferase (ALT) and aspartate aminotransferase (AST) with no signs of kidney structural or functional damage (Table 4). Galactosamine treatment did not lead to any increase in mRNA levels of Fgα, Fgβ and Fgγ in the kidney as well as urinary excretion of Fg, suggesting increased urinary Fg to be a specific indication of kidney damage (Table 4).

TABLE 4
mRNA expression of Fgα, Fgβ, and Fgγ and urinary excretion of
fibrinogen does not increase following galactosamine-induced liver toxicity

| | Galactosamine | |
|---|---|---|
| | 0 g/kg | 1.1 g/kg |
| (a) histopathological score | | |
| kidney | 0 | 0 |
| liver | 0 | 2 |
| (b) clinical chemistry | | |
| BUN [mg/dl] | 19.9 ± 0.8 | 19.1 ± 1.1 |
| Scr [mg/dl] | 0.5 ± 0.0 | 0.5 ± 0.0 |
| ALT [U/l] | 26.0 ± 1.9 | 422.8 ± 121.9* |
| AST [U/l] | 42.5 ± 3.9 | 246.0 ± 57.5 |
| (c) gene expression (FC relative to control) | | |
| Fgα | 1.0 ± 0.2 | 1.2 ± 0.1 |
| Fgβ | 1.0 ± 0.1 | 1.0 ± 0.0 |
| Fgγ | 1.0 ± 0.1 | 0.9 ± 0.1 |
| Kim-1 | 1.0 ± 0.1 | 1.0 ± 0.1 |
| (d) urinary excretion | | |
| Fg [ng/mg Cr] | 201.9 ± 38.1 | 26.0 ± 26.0* |

Data are presented as mean ± SEM. Statistical analysis was performed by Student's t-test; *p < 0.05

### Example 9

### Increased Urinary Fibrinogen in Patients with Postoperative Acute Kidney Injury Following Abdominal Aortic Aneurysm

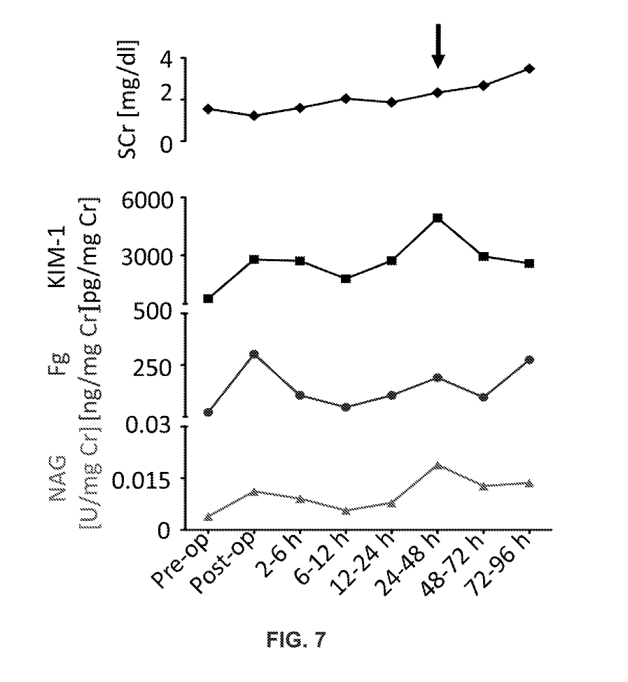

Of 31 patients undergoing AAA repair, 7 developed postoperative AKI as defined as ≧50% rise in SCr. Demographic characteristics of patients according to AKI status are shown in Table 5. Among the 7 patients with postoperative AKI, serum creatinine levels tended to rise to ≧50% of baseline values within 48 h after surgery whereas urinary Fg, KIM-1, and NAG showed earlier rises (FIG. 7). The diagnostic ability of Fg, KIM-1 and NAG to identify AKI versus no-AKI is shown in Table 6. AUC-ROC exceeded 0.7 for each biomarker at different time points (24-48 h and 48-72 h for Fg; 2-6 h for KIM-1; and 6-12 h for NAG).

TABLE 5
Demographic and clinical characteristics

| | AKI (n = 7) | no-AKI (n = 24) |
|---|---|---|
| Age | 76.7 ± 6.9 | 71.4 ± 9.5 |
| Female, % | 42.9 | 37.5 |
| Diabetes, % | 14.3 | 25 |
| Hypertension, % | 100 | 91.7 |
| Juxta-or supra-renal, % | 28.6 | 33.3 |
| Pre-op SCr | 1.45 ± 0.7 | 1.1 ± 0.4 |
| Pre-op GFR | 58.7 ± 37.5 | 62.2 ± 19.4 |
| Weight, kg | 90.1 ± 12.8 | 81.5 ± 20.6 |
| Cross-clamp time, min | 80.9 ± 27.3 | 48.9 ± 19.9 |

TABLE 6
Comparative diagnostic performance characteristics of urinary
biomarkers for the identification of established AKI using the area under
the receiver operating characteristics curve (AUC-ROC)

| | | | | # of patients |
|---|---|---|---|---|
| | AUC (95% CI) | | | (AKI and no |
| | Fg | KIM-1 | NAG | AKI) |
| Pre-op | 0.56 | 0.520 | 0.55 | 7, 22 |
| | (0.36-0.74) | (0.33-0.71) | (0.36-0.73) | |
| Post op | 0.680 | 0.66 | 0.62 | 6, 23 |
| | (0.48-0.84) | (0.46-0.82) | (0.42-0.79) | |
| 2-6 h | 0.63 | 0.75 | 0.51 | 5, 20 |
| | (0.42-0.81) | (0.54-0.90) | (0.30-0.71) | |
| 6-12 h | 0.520 | 0.58 | 0.75 | 4, 12 |
| | (0.26-0.77) | (0.32-0.82) | (0.48-0.93) | |
| 12-24 h | 0.65 | 0.64 | 0.52 | 6, 20 |
| | (0.44-0.83) | (0.43-0.82) | (0.31-0.72) | |
| 24-48 h | 0.72 | 0.550 | 0.610 | 7, 24 |
| | (0.53-0.87) | (0.37-0.73) | (0.42-0.78) | |
| 48-72 h h | 0.7 | 0.520 | 0.510 | 6, 24 |
| | (0.51-0.85) | (0.34-0.71) | (0.32-0.70) | |
| 72 h-96 h | 0.61 | 0.51 | 0.45 | 5, 21 |
| | (0.40-0.79) | (0.30-0.71) | (0.32-0.74) | |

### Example 10

### Immunostaining Patterns of Fibrinogen as an Indicator of Kidney Tubular Damage in Patients with Biopsy-Proven Acute Tubular Injury

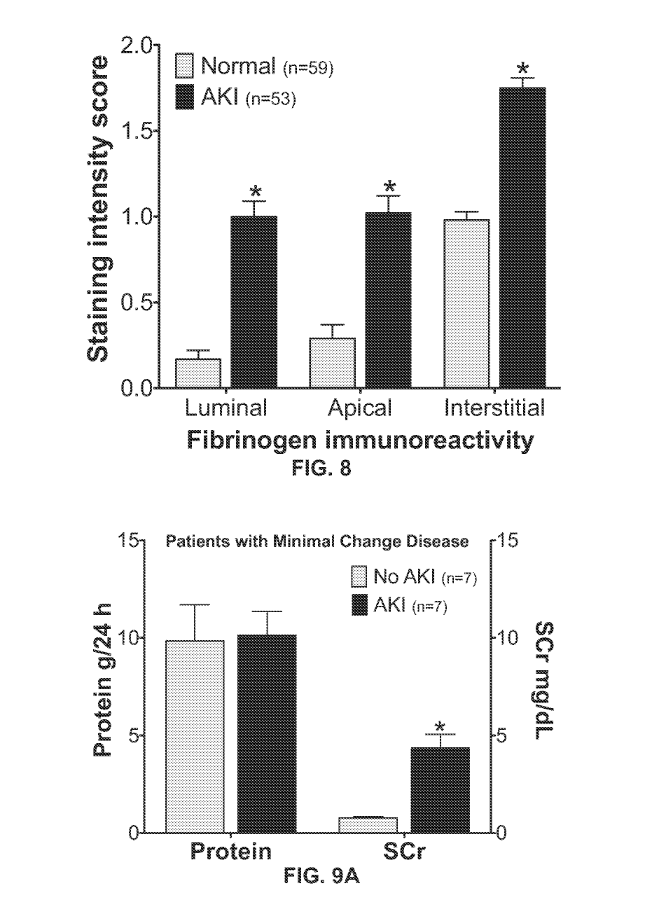

Morphological diagnosis of acute tubular necrosis (ATN) in human kidney biopsy samples was established based on light microscopic findings. In the absence of kidney injury, the tubules were closely packed together, “back-to-back” and revealed preserved cellular details, while a low epithelial lining and dilatation of the tubules, with accumulation of necrotic cells and cellular debris in their lumens, as well as mild interstitial edema was observed in all cases of acute tubular injury. Immunoreactivity for fibrinogen/fibrin-related antigens was examined in all compartments of kidney parenchyma including glomeruli, tubules, interstitium, and vasculature. There was no evidence of fibrinogen immunoreactivity in the glomerulus or vasculature; in particular, signs of vascular or glomerular thrombosis or segmental fibrinoid necrosis were absent in any of the examined cases. A distinctly differential immunostaining pattern in the apical and luminal region of the tubules was noted in patients with (n=53) or without ATN (n=59). Fine granular reactivity in the apical region of the tubular epithelial cell cytoplasm was noted that became much more pronounced and widespread in the ATN patients sometimes but not necessarily in association with tubular distension and/or luminal staining (FIG. 8). Luminal staining was characterized by the reactivity of accumulated intraluminal fibrinogen that usually consists of cellular debris admixed with proteinaceous material. There was a significant increase in luminal immunoreactivity of fibrinogen in the ATN patients as compared to the normal (FIG. 8). The interstitial staining for fibrinogen was noted in the vast majority of the biopsies and also showed increased immunoreactivity in the AKI patients as compared to normal (FIG. 8).

### Example 11

### Immunostaining Patterns of Fibrinogen Differentiate Patients With Minimal Change Disease (MCD) that Develop AKI from MCD Patients that do not Develop AKI

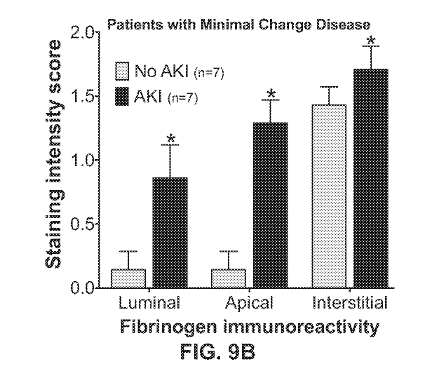

The samples from the patients with minimal change disease (MCD) did not reveal significant glomerular pathology by light microscopy, but they all demonstrated diffuse effacement of visceral epithelial cell foot processes on electron microscopy; seven patients also demonstrated signs of acute tubular injury (FIG. 9A). Accordingly, all patients presented with nephrotic syndrome and prominent proteinuria (9.86±1.84 g/24 h vs 10.14±1.22 g/24 h, FIG. 9A) but some were associated acute renal failure as indicated by serum creatinine (0.77±0.07 mg/dL vs. 4.35±0.71, FIG. 9A). Fibrinogen immunoreactivity was significantly increased in the luminal, apical, and interstitial regions (FIG. 9B) in MCD patients that developed AKI as compared to MCD patients that did not develop AKI. Approximately 6-fold increase in luminal staining, 9-fold increase in apical staining and 1.5 fold increase in interstitial immunoreactivity of fibrinogen in MCD patients with AKI as compared to MCD patients without AKI suggests that expression patterns of fibrinogen immunostaining in the kidney can serve as an effective way to diagnose AKI in MCD patients.
1. Kant J A, Formace A J, Jr., Saxe D, Simon M I, McBride O W, Crabtree G R. Evolution and organization of the fibrinogen locus on chromosome 4: gene duplication accompanied by transposition and inversion. Proc Natl Acad Sci U S A. 1985; 82(8):2344-2348. Prepublished on 1985 Apr. 1 as DOI.
2. Molmenti E P, Ziambaras T, Perlmutter D H. Evidence for an acute phase response in human intestinal epithelial cells. J Biol Chem. 1993; 268(19):14116-14124. Prepublished on 1993 Jul. 5 as DOI.
3. Lee S Y, Lee K P, Lim J W. Identification and biosynthesis of fibrinogen in human uterine cervix carcinoma cells. Thromb Haemost. 1996; 75(3):466-470. Prepublished on 1996 Mar. 1 as DOI.
4. Simpson-Haidaris P J, Courtney M A, Wright T W, Goss R, Harmsen A, Gigliotti F. Induction of fibrinogen expression in the lung epithelium during Pneumocystis carinii pneumonia. Infect Immun. 1998; 66(9):4431-4439. Prepublished on 1998 Aug. 26 as DOI.
5. Simpson-Haidaris P J, Wright T W, Earnest B J, Hui Z, Neroni L A, Courtney M A. Cloning and characterization of a lung-specific cDNA corresponding to the gamma chain of hepatic fibrinogen. Gene. 1995; 167(1-2):273-278. Prepublished on 1995 Dec. 29 as DOI.
6. Lord S T. Fibrinogen and fibrin: scaffold proteins in hemostasis. Curr Opin Hematol. 2007; 14(3):236-241. Prepublished on 2007 Apr. 7 as DOI 10.1097/MOH.0b013e3280dce58c00062752-200705000-00010 [pii].
7. Degen M. Hemostatic factors and inflammatory disease. Thromb Haemost. 1999; 82(2):858-864. Prepublished on 1999 Dec. 22 as DOI 99080858 [pii].
8. Drew A F, Liu H, Davidson J M, Daugherty C C, Degen M. Wound-healing defects in mice lacking fibrinogen. Blood. 2001; 97(12):3691-3698. Prepublished on 2001 Jun. 5 as DOI.
9. Palumbo J S, Degen J L. Hemostatic factors in tumor biology. J Pediatr Hematol Oncol. 2000; 22(3):281-287. Prepublished on 2000 Jun. 23 as DOI.
10. Palumbo J S, Kombrinck K W, Drew A F, et al. Fibrinogen is an important determinant of the metastatic potential of circulating tumor cells. Blood. 2000; 96(10):3302-3309. Prepublished on 2000 Nov. 9 as DOI.
11. Adams R A, Schachtrup C, Davalos D, Tsigelny I, Akassoglou K. Fibrinogen signal transduction as a mediator and therapeutic target in inflammation: lessons from multiple sclerosis. Curr Med Chem. 2007; 14(27):2925-2936. Prepublished on 2007 Nov. 30 as DOI.
12. Petri A L, Simonsen A H, Yip T T, et al. Three new potential ovarian cancer biomarkers detected in human urine with equalizer bead technology. Acta Obstet Gynecol Scand. 2009; 88(1):18-26. Prepublished on 2008 Nov. 22 as DOI 905768664 [pii] 10.1080/00016340802443830.
13. Lovely R S, Kazmierczak S C, Massaro J M, D'Agostino R B, O'Donnell C J, Farrell D H. ‘Fibrinogen: Evaluation of a New Assay for Study of Associations with Cardiovascular Disease. Clin Chem. 2010; 56(5):781-788. 10.1373/clinchem. 2009.138347.
14. Thomson N M, Moran J, Simpson I J, Peters D K. Defibrination with ancrod in nephrotoxic nephritis in rabbits. Kidney Int. 1976; 10(5):343-347. Prepublished on 1976 Nov. 1 as DOI.
15. Dempfle C E, Argiriou S, Kucher K, Muller-Peltzer H, Rubsamen K, Heene D L. Analysis of fibrin formation and proteolysis during intravenous administration of ancrod. Blood. 2000; 96(8):2793-2802. Prepublished on 2000 Oct. 7 as DOI.
16. Cheresh D A, Berliner S A, Vicente V, Ruggeri Z M. Recognition of distinct adhesive sites on fibrinogen by related integrins on platelets and endothelial cells. Cell. 1989; 58(5):945-953. Prepublished on 1989 Sep. 8 as DOI 0092-8674(89)90946-X [pii].
17. Languino L R, Plescia J, Duperray A, et al. Fibrinogen mediates leukocyte adhesion to vascular endothelium through an ICAM-1-dependent pathway. Cell. 1993; 73(7):1423-1434. Prepublished on 1993 Jul. 2 as DOI 0092-8674(93)90367-Y [pii].
18. Ryu J K, Davalos D, Akassoglou K. Fibrinogen signal transduction in the nervous system. Journal of Thrombosis and Haemostasis. 2009; 7:151-154. 10.1111/j.1538-7836.2009.03438.x.
19. Arrizabalaga P, Sole M, Abellana R, et al. Tubular and interstitial expression of ICAM-1 as a marker of renal injury in IgA nephropathy. Am J Nephrol. 2003; 23(3):121-128. Prepublished on 2003 Feb. 5 as DOI 10.1159/000068920 AJN23121 [pii].
20. Kim S, Nadel J A. Fibrinogen binding to ICAM-1 promotes EGFR-dependent mucin production in human airway epithelial cells. Am J Physiol Lung Cell Mol Physiol. 2009; 297(1):L174-183. Prepublished on 2009 May 12 as DOI 00032.2009 [pii] 10.1152/ajplung.00032.2009.
21. Simon E E. Potential role of integrins in acute renal failure. Nephrol Dial Transplant. 1994; 9 Suppl 4:26-33. Prepublished on 1994 Jan. 1 as DOI.
22. Warnock D G, Peck C C. A roadmap for biomarker qualification. Nat Biotechnol 2010; 28: 444-445.
23. Goodsaid F M, Mendrick D L. Translational medicine and the value of biomarker qualification. Sci Transl Med 2010; 2: 47 ps44.
24. Zhou Y, Vaidya V S, Brown R P, et al. Comparison of kidney injury molecule-1 and other nephrotoxicity biomarkers in urine and kidney following acute exposure to gentamicin, mercury, and chromium. Toxicol Sci 2008; 101: 159-170.
25. Waikar S S, Liu K D, Chertow G M. The incidence and prognostic significance of acute kidney injury. Curr Opin Nephrol Hypertens 2007; 16: 227-236.
26. Waikar S S, Winkelmayer W C. Chronic on acute renal failure: long-term implications of severe acute kidney injury. JAMA 2009; 302: 1227-1229.
27. Winterberg P D, Lu C Y. Acute Kidney Injury: The Beginning of the End of the Dark Ages. Am J Med Sci 2011.
28. Harpur E, Ennulat D, Hoffman D, et al. Biological Qualification of Biomarkers of Chemical-Induced Renal Toxicity in Two Strains of Male Rat. Toxicol Sci 2011; 122: 235-252.
29. Parikh C R, Coca S G, Thiessen-Philbrook H, et al. Postoperative Biomarkers Predict Acute Kidney Injury and Poor Outcomes after Adult Cardiac Surgery. J Am Soc Nephrol 2011.
30. Shlipak M G, Coca S G, Wang Z, et al. Presurgical Serum Cystatin C and Risk of Acute Kidney Injury After Cardiac Surgery. Am J Kidney Dis 2011.
31. Doolittle R F. Fibrinogen and fibrin. Annu Rev Biochem 1984; 53: 195-229.
32. Weisel J W. Fibrinogen and fibrin. Adv Protein Chem 2005; 70: 247-299.
33. Adams R A, Schachtrup C, Davalos D, et al. Fibrinogen signal transduction as a mediator and therapeutic target in inflammation: lessons from multiple sclerosis. Curr Med Chem 2007; 14: 2925-2936.
34. Schultz D R, Arnold P I. Properties of four acute phase proteins: C-reactive protein, serum amyloid A protein, alpha 1-acid glycoprotein, and fibrinogen. Semin Arthritis Rheum 1990; 20: 129-147.
35. Sabbahy M E, Vaidya V S. Ischemic kidney injury and mechanisms of tissue repair. Wiley Interdiscip Rev Syst Biol Med 2011; 3: 606-618.
36. Bonventre J V. Pathophysiology of acute kidney injury: roles of potential inhibitors of inflammation. Contrib Nephrol 2007; 156: 39-46.
37. Pabla N, Dong Z. Cisplatin nephrotoxicity: mechanisms and renoprotective strategies. Kidney Int 2008; 73: 994-1007.
38. Danesh J, Collins R, Appleby P, et al. Association of fibrinogen, C-reactive protein, albumin, or leukocyte count with coronary heart disease: meta-analyses of prospective studies. JAMA 1998; 279: 1477-1482.
39. Catto A J, Grant P J. Risk factors for cerebrovascular disease and the role of coagulation and fibrinolysis. Blood Coagul Fibrinolysis 1995; 6: 497-510.
40. van Oijen M, Witteman J C, Hofman A, et al. Fibrinogen is associated with an increased risk of Alzheimer disease and vascular dementia. Stroke 2005; 36: 2637-2641.
41. Prinsen B H, Rabelink T J, Beutler J J, et al. Increased albumin and fibrinogen synthesis rate in patients with chronic renal failure. Kidney Int 2003; 64: 1495-1504.
42. Parikh C R, Coca S G, Thiessen-Philbrook H, et al. Postoperative Biomarkers Predict Acute Kidney Injury and Poor Outcomes after Adult Cardiac Surgery. Am Soc Nephrol 2011; 22: 1748-1757.
43. Siew E D, Ware L B, Gebretsadik T, et al. Urine neutrophil gelatinase-associated lipocalin moderately predicts acute kidney injury in critically ill adults. J Am Soc Nephrol 2009; 20: 1823-1832.
44. Waikar S S, Betensky R A, Bonventre T V. Creatinine as the gold standard for kidney injury biomarker studies? Nephrol Dial Transplant 2009; 24: 3263-3265.
45. Waikar S S, Bonventre T V. Creatinine kinetics and the definition of acute kidney injury. J Am Soc Nephrol 2009; 20: 672-679.
46. Waldman M, Crew R J, Valeri A, et al. Adult minimal-change disease: clinical characteristics, treatment, and outcomes. Clin J Am Soc Nephrol 2007; 2: 445-453.
47. Jennette J C, Falk R J. Adult minimal change glomerulopathy with acute renal failure. Am J Kidney Dis 1990; 16: 432-437.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of detecting the presence of acute kidney injury (AM) in a subject, the method comprising:
determining a level of fibrinogen in a sample comprising urine from a subject; and
comparing the level of fibrinogen in the sample to a reference level of fibrinogen,
wherein the level of fibrinogen as compared to the reference level indicates whether the subject has AM.

2. The method of claim 1, further comprising selecting a subject who is suspected of or at risk for having AM, or who has one or more risk factors for developing AM.

3. The method of claim 1, wherein determining a level of fibrinogen comprises determining a level of whole fibrinogen protein, and/or one, two, or all of the α, β and γ chains of fibrinogen.

4. The method of claim 1, wherein the reference level represents a level of fibrinogen in a subject who does not have AM, and the presence of a level of fibrinogen above the reference level indicates that the subject has AM.

5. The method of claim 1, further comprising administering a treatment for AM to a subject who has a level of fibrinogen above the reference level.

6. A method of treating a subject, the method comprising:
determining a level of fibrinogen in a sample comprising urine of a subject;
comparing the level of fibrinogen in the sample to a reference level of fibrinogen; and
selecting a subject who has a level of fibrinogen that is above the reference level; and
administering a treatment for acute kidney injury (AKI) to the selected subject.

7. The method of claim 6, further comprising selecting a subject who is suspected of or at risk for having AM, or who has one or more risk factors for developing AM.

8. The method of claim 6, wherein determining a level of fibrinogen comprises determining a level of whole fibrinogen protein, and/or one, two, or all of the α, β and γ chains of fibrinogen.

9. The method of claim 6, wherein the reference level represents a level of fibrinogen in a subject who does not have AM, and the presence of a level of fibrinogen above the reference level indicates that the subject has AM.

10. (canceled)

11. (canceled)

12. A method of treating a subject who has acute kidney injury, the method comprising administering a composition comprising a therapeutically effective amount of peptide comprising Bβ₁₅₋₄₂ (GHRPLDKKREEAPSLRPAPPPISGGGYR (SEQ ID NO:31)), to a subject who is in need of, or who has been determined to be in need of, such treatment.

13. The method of claim 5, wherein the subject has been identified as having AM.

14. The method of claim 12, wherein the peptide comprises Bβ₁₅₋₄₂ peptide fused to a cell-penetrating peptide.
